(19) United States
(12) Patent Application Publication
(10) Pub. No.: US 2010/0285051 A1
(43) Pub. Date: Nov. 11, 2010

(54) VACCINE

(76) Inventors: Dominique Ingrid Lemoine, Rixensart (BE); Sophie Valerie Anne Ponsard, Rixensart (BE)

Correspondence Address: GlaxoSmithKline;GLOBAL PATENTS -US, UW2220, P. O. BOX 1539, KING OF PRUSSIA, PA, 19406-0939, US

(21) Appl. No.: 12/809,774

(22) PCT Filed: Dec. 18, 2008
(86) PCT No.: PCT/EP2008/067945

§ 371 Date: Jun. 21, 2010

Related U.S. Application Data
(60) Provisional application No. 61/015,767, filed on Dec. 21, 2007.
(60) Provisional application No. 61/019,951, filed on Jan. 9, 2008.

(51) Int. Cl. A61K 39/21; A61P 31/18

(52) U.S. Cl. 424/192.1

(57) ABSTRACT

A component for a HIV vaccine comprising:
a) an immunogenic fusion protein comprising Nef or an immunogenic fragment or derivative thereof, and p17 Gag and/or p24 Gag or immunogenic fragments or derivatives thereof, wherein when both p17 and p24 Gag are present there is at least one HIV antigen or immunogenic fragment between them, and
b) a stabilising agent selected from the group comprising or consisting of monothioglycerol, cysteine, N-acetyl cysteine or mixtures thereof

The invention also extends to HIV vaccines comprising the same and use in treatment/prevention of HIV.

### FIELD OF THE INVENTION

The present invention relates to novel compositions comprising a HIV fusion protein, in particular the HIV fusion protein referred to herein as F4, and a stabilizing agent; methods of preparing the same and use in the treatment and/or prevention of HIV-1 infection and/or acquired immune deficiency syndrome AIDS.

HIV-1 is the primary cause of the AIDS which is regarded as one of the world's major health problems. There is a need for a vaccine for the prevention and/or treatment of HIV infection.

### BACKGROUND TO THE INVENTION

HIV-1 is an RNA virus of the family Retroviridiae. The HIV genome encodes at least nine proteins which are divided into three classes: the major structural proteins Gag, Pol and Env, the regulatory proteins Tat and Rev, and the accessory proteins Vpu, Vpr, Vif and Nef. The HIV genome exhibits the 5′LTR-gag-pol-env-LTR3′ organization of all retroviruses.

The HIV envelope glycoprotein gp120 is the viral protein that is used for attachment to the host cell. This attachment is mediated by binding to two surface molecules of helper T cells and macrophages, known as CD4 and one of the two chemokine receptors CCR-5 or CXCR-4. The gp120 protein is first expressed as a larger precursor molecule (gp160), which is then cleaved post-translationally to yield gp120 and gp41. The gp 120 protein is retained on the surface of the virion by linkage to the gp41 molecule, which is inserted into the viral membrane.

The gp120 protein is the principal target of neutralizing antibodies, but unfortunately the most immunogenic regions of the proteins (V3 loop) are also the most variable parts of the protein. Therefore, the use of gp120 (or its precursor gp160) as a vaccine antigen to elicit neutralizing antibodies is thought to be of limited use for a broadly protective vaccine. The gp120 protein does also contain epitopes that are recognized by cytotoxic T lymphocytes (CTL). These effector cells are able to eliminate virus-infected cells, and therefore constitute a second major antiviral immune mechanism. In contrast to the target regions of neutralizing antibodies some CTL epitopes appear to be relatively conserved among different HIV strains. For this reason gp120 and gp160 maybe useful antigenic components in vaccines, for example containing a cocktail of antigens/components, that aim at eliciting cell-mediated immune responses (particularly CTL).

Non-envelope proteins of HIV-1 include for example internal structural proteins such as the products of the Gag and pol genes and other non-structural proteins such as Rev, Nef, Vif and Tat (Green et al., New England J. Med, 324, 5, 308 et seq (1991) and Bryant et al. (Ed. Pizzo), Pediatr. Infect. Dis. J., 11, 5, 390 et seq (1992).

HIV Nef is expressed early in infection and in the absence of structural protein.

The Nef gene encodes an early accessory HIV protein which has been shown to possess several activities. For example, the Nef protein is known to cause the down regulation of CD4, the HIV receptor, and MHC class I molecules from the cell surface, although the biological importance of these functions is debated. Additionally Nef interacts with the signal pathway of T cells and induces an active state, which in turn may promote more efficient gene expression. Some HIV isolates have mutations in this region, which cause them not to encode functional protein and are severely compromised in their replication and pathogenesis in vivo.

The Gag gene is translated as a precursor polyprotein that is cleaved by proteases to yield products that include the matrix protein (p17), the capsid (p24), the nucleocapsid (p9), p6 and two space peptides, p2 and p1.

The Gag gene gives rise to the 55-kilodalton (kD) Gag precursor protein, also called p55, which is expressed from the unspliced viral mRNA. During translation, the N-terminus of p55 is myristoylated, triggering its association with the cytoplasmic aspect of cell membranes. The membrane-associated Gag polyprotein recruits two copies of the viral genomic RNA along with other viral and cellular proteins that triggers the budding of the viral particle from the surface of an infected cell. After budding, p55 is cleaved by the virally encoded protease (a product of the pol gene) during the process of viral maturation into four smaller proteins designated MA (matrix [p17]), CA (capsid [p24]), NC (nucleocapsid [p9]), and p6.

In addition to the 3 major Gag proteins, all Gag precursors contain several other regions, which are cleaved out and remain in the virion as peptides of various sizes. These proteins have different roles e.g. the p2 protein has a proposed role in regulating activity of the protease and contributes to the correct timing of proteolytic processing.

The p17 (MA) polypeptide is derived from the N-terminal, myristoylated end of p55. Most MA molecules remain attached to the inner surface of the virion lipid bilayer, stabilizing the particle. A subset of MA is recruited inside the deeper layers of the virion where it becomes part of the complex which escorts the viral DNA to the nucleus. These MA molecules facilitate the nuclear transport of the viral genome because a karyophilic signal on MA is recognized by the cellular nuclear import machinery. This phenomenon allows HIV to infect non-dividing cells, an unusual property for a retrovirus.

The p24 (CA) protein forms the conical core of viral particles. Cyclophilin A has been demonstrated to interact with the p24 region of p55 leading to its incorporation into HIV particles. The interaction between Gag and cyclophilin A is essential because the disruption of this interaction by cyclosporin A inhibits viral replication.

The NC region of Gag is responsible for specifically recognizing the so-called packaging signal of HIV. The packaging signal consists of four stem loop structures located near the 5′ end of the viral RNA, and is sufficient to mediate the incorporation of a heterologous RNA into HIV-1 virions. NC binds to the packaging signal through interactions mediated by two zinc-finger motifs. NC also facilitates reverse transcription.

The p6 polypeptide region mediates interactions between p55 Gag and the accessory protein Vpr, leading to the incorporation of Vpr into assembling virions. The p6 region also contains a so-called late domain which is required for the efficient release of budding virions from an infected cell.

The Pol gene encodes two proteins containing the two activities needed by the virus in early infection, the RT and the integrase protein needed for integration of viral DNA into cell DNA. The primary product of Pol is cleaved by the virion protease to yield the amino terminal RT peptide which contains activities necessary for DNA synthesis (RNA and DNA-dependent DNA polymerase activity as well as an RNase H function) and carboxy terminal integrase protein. HIV RT is a heterodimer of full-length RT (p66) and a cleavage product (p51) lacking the carboxy terminal RNase H domain.

RT is one of the most highly conserved proteins encoded by the retroviral genome. Two major activities of RT are the DNA Pol and Ribonuclease H. The DNA Pol activity of RT uses RNA and DNA as templates interchangeably and like all DNA polymerases known is unable to initiate DNA synthesis de novo, but requires a pre-existing molecule to serve as a primer (RNA).

The RNase H activity inherent in all RT proteins plays the essential role early in replication of removing the RNA genome as DNA synthesis proceeds. It selectively degrades the RNA from all RNA-DNA hybrid molecules. Structurally the polymerase and ribo H occupy separate, non-overlapping domains with the Pol covering the amino two thirds of the Pol.

The p66 catalytic subunit is folded into 5 distinct subdomains. The amino terminal 23 of these have the portion with RT activity. Carboxy terminal to these is the RNase H Domain.

WO 2006/013106 describes fusion proteins which comprises Nef or an immunogenic fragment or derivative thereof, and p17 Gag and/or p24 Gag or immunogenic fragments or derivatives thereof, wherein when both p17 and p24 Gag are present there is at least one HIV antigen or immunogenic fragment between them. In one embodiment the fusion protein is named F4.

The proteins of this type, in particular F4, are sensitive to precipitation, aggregation, pH, light, agitation, adsorption and/or oxidation. This may be true even when the antigen is lyophilized for storage for subsequent reconstitution with, for example liquid adjuvant just before use. These phenomena in particular precipitation, aggregation and/or oxidation may result in loss of advantageous biological properties such as immunogenicity and/or antigenicity or may result in giving the formulation other undesirable properties. Furthermore, pharmaceutical products for human use must be well characterized, stable and safe.

Thiomersal has been used as a preservative to avoid growth of microbial organisms in certain formulations and sodium sulfite has been used to stabilise certain antigens. However, there are disadvantages associated with the above reagents, in particular some formulators prefer not to use thiomersal because they desire to exclude mercury containing compounds in vaccines. Sodium sulfite is thought to have the potential to cause allergic reactions from some individuals. Therefore, if sodium sulfite is included in the formulation then a warning may be required on the label as the formulation may not be suitable for use in all individuals.

The inventors investigated the addition of agents such as citric acid trisodium salt, malic acid sodium salt, dextrose and L-methionine to the formulation but these did not have the desired effect. Nevertheless the inventors have now found that said proteins particularly F4 can be stabilize without use of sodium sulfite.

### SUMMARY OF THE INVENTION

Thus the invention provides bulk formulation or a component for a HIV vaccine comprising:
a) an immunogenic fusion protein comprising Nef or an immunogenic fragment or derivative thereof, and p17 Gag and/or p24 Gag or immunogenic fragments or derivatives thereof, wherein when both p17 and p24 Gag are present there is at least one HIV antigen or immunogenic fragment between them, and
b) a stabilising agent which is an antioxidant containing a thiol functional group for example selected from the group consisting of glutathione, monothioglycerol, cysteine, N-acetyl cysteine or mixtures thereof.

### BRIEF DESCRIPTION OF THE FIGURES

FIG. 1 Shows SDS-PAGE analysis under reducing conditions of F4

FIG. 2 Shows solubility assays for F4

FIG. 3 Shows Coomassie stained gel & Western Blot for codon-optimized F4

FIG. 4 Shows Coomassie stained gel & Western Blot for codon-optimized p51RT

FIG. 5 Shows solubility assays for RT/p55 and RT/p66

FIG. 6 Shows SDS-PAGE analysis under reducing conditions for various F4 proteins

FIG. 7 Shows SDS-PAGE follow up of the purification of F4co and carboxyamidated F4co. 5 μg of each fraction collected during the purification of F4co or F4coca were separated on a 4-12% SDS gel. The gel was Coomassie blue stained. 1: Homogenate; 2: CM hyperZ eluate; 3: Q sepharose eluate; 4: Purified bulk

FIG. 8 SDS-PAGE analysis of F4, F4co and F4coca purified according to purification method I or method II. 5 μg of each protein were separated on a 4-12% SDS gel in reducing conditions (left) or non-reducing conditions (right). The gel was Coomassie blue stained. 1: Method II—F4co; 2: Method II—F4coca; 3: Method I—F4coca; 4: Method I—F4; 5: Method I—F4 carboxyamidated

FIG. 9 Screening of chelating agents by SDS PAGE under non-reducing conditions

FIG. 10 SDS-PAGE under non reducing conditions of FINAL BULK stability T15 days 4° C. of the formulations containing glutathione and monothioglycerol

FIG. 11 SDS-PAGE under non reducing conditions of FINAL BULK stability T15 days 4° C. of the formulations containing cysteine and acetyl cysteine

FIG. 12 SDS-PAGE in non reducing conditions of reconstituted lyophilized antigen (cakes) containing glutathione and monothioglycerol

FIG. 13 SDS-PAGE in non reducing conditions of reconstituted cakes containing cysteine and acetylcysteine

FIG. 14 SDS-PAGE analysis under reducing conditions of reconstituted cakes containing cysteine and acetylcysteine in liposomal ajuvant containing MPL and QS21 after 4 hours at 25 degrees C. (before and after centrifugation)

### DETAILED DESCRIPTION OF THE INVENTION

Advantageously, use of at least stabilizing agent monothioglycerol or N-acetyl cysteine listed above in part b) in accordance with the invention is thought to provide equivalent or better stabilization than sodium sulfite. That is to say when sodium sulfite is employed to stabilize said proteins/antigens intramolecular oxidation, seems to be quenched but some aggregation, thought to be due to intermolecular oxidation is observed (ie by formation of disulfide bonds between molecules). In contrast when the one or more of monothioglycerol, cysteine or N-acetyl cysteine is employed at the appropriate level, then no aggregation is observed thereby providing better stabilization than sodium sulfite. Furthermore, the solubility of the antigen is maintained/retained.

Whilst not wishing to be bound by theory, it is thought that the thiol functionality in the antioxidant either links to thiol groups in the protein and/or oxidizes preferentially thereby preventing oxidation in the protein.

Furthermore the desirable properties of the protein such as immunogenicity and/or antigenicity and the like may be maintained in formulations of the invention.

In one aspect the stabilizing agent is monothioglycerol.

In one aspect the stabilizing agent is cyteine.

In one aspect the stabilizing agent is N-acetyl cysteine.

In one aspect the stabilizing agent is glutathione.

In at least one aspect the final bulk or liquid formulation is substantially free of alkali metal sulfite, such as sodium sulfite.

In another aspect the final bulk or liquid formulation is substantially free of thiomersal.

The stabilizing agent may be present in amounts in the range 0.001-2.5% w/v, such as 0.01, 0.1, 0.2, 0.3, 0.4, 0.5, 0.6, 0.7, 0.8, 0.9% or 1 w/v, particularly 0.5% w/v.

The antioxidants solutions may be prepared as follows:
Powder or liquid weighing
Dissolution in water for injection, for example about 80 ml
Addition of water to predefined limit, for example till 100 ml
pH adjustment with NaOH 1M, for example to about pH7.5

In the constructs employed in the invention and compositions according to the invention as described herein, the Nef may be a full length Nef.

In one embodiment the Nef is non-myristolylated.

In the constructs employed in the invention the p17 Gag and p24 Gag are, for example, full length p17 and p24 respectively.

In one embodiment the polypeptide employed comprises both p17 and p24 Gag or immunogenic fragments thereof. In such a construct the p24 Gag component and p17 Gag component are separated by at least one further HIV antigen or immunogenic fragment, such as Nef and/or RT or immunogenic fragments or derivatives thereof.

Alternatively p17 or p24 Gag may be provided separately.

In another embodiment the polypeptide construct employed in the invention further comprises Pol or a derivative of Pol such as RT or an immunogenic fragment or derivative thereof. Particular fragments of RT that are suitable for use in the invention are fragments in which the RT is truncated at the C terminus, for example such that they lack the carboxy terminal RNase H domain. One such fragment lacking the carboxy terminal Rnase H domain is the p51 fragment described herein.

The RT or immunogenic fragment in the fusion proteins described herein may, for example be p66 RT or p51 RT.

The RT component of the fusion protein or composition employed in the invention optionally comprises a mutation at position 592, or equivalent mutation in strains other than HXB2, such that the methionine is removed by mutation to another residue e.g. lysine. The purpose of this mutation is to remove a site which serves as an internal initiation site in prokaryotic expression systems.

The RT component also, or alternatively, may comprise a mutation to remove the enzyme activity (reverse transcriptase). Thus K231 may be present instead of W.

In fusion proteins employed in the invention which comprise p24 and RT, it may be advisable to employ a construct where p24 precedes the RT because when the antigens are expressed alone in E. coli better expression of p24 than of RT is observed.

Particular constructs according to the invention include the following:

1. p24-RT-Nef-p17 (also referred to herein as F4)

2. p24-RT*-Nef-p17

3. p24 -p51RT-Nef-p17

4. p24-p51RT*-Nef-p17

* represents RT methionine₅₉₂ mutation to lysine

In one aspect the fusion protein is F4.

In a further aspect of the invention the F4 or other fusion protein employed may be chemically treated to assist purification and/or to retain desirable biological properties.

Suitable chemical treatments include carboxymethylation, carboxyamidation, acetylation or treatment with an aldehyde such as formaldehyde or glutaldehyde.

In one aspect the fusion protein is F4co, wherein the polynucleotide encoding said protein or part thereof has been codon-optimized.

An immune response may be measured by a suitable immunological assay such as an ELISA (for antibody responses) or flow cytometry using suitable staining for cellular markers and cytokines (for cellular responses).

The polypeptide constructs of HIV antigens employed in the invention are capable of being expressed in in vitro systems including prokaryotic systems such as E. coli. Advantageously they can be purified by conventional purification methods.

The fusions described herein may be soluble when expressed in a selected expression system, that is they are present in a substantial amount in the supernatant of a crude extract from the expression system. The presence of the fusion protein in the crude extract can be measured by conventional means such as running on an SDS gel, coomassie staining and checking the appropriate band by densitometric measurement. Fusion proteins according to the invention are for example at least 50% soluble, such as at least 70% soluble, particularly 90% soluble or greater as measured by the techniques described herein in the Examples. Techniques to improve solubility of recombinantly expressed proteins are known, for example in prokaryotic expression systems solubility is improved by lowering the temperature at which gene expression is induced.

Immunogenic fragments as described herein will contain at least one epitope of the antigen and display HIV antigenicity and are capable of raising an immune response when presented in a suitable construct, such as for example when fused to other HIV antigens or presented on a carrier, the immune response being directed against the native antigen. Typically the immunogenic fragments contain at least 20, for example 50, such as 100 contiguous amino acids from the HIV antigen.

The component may be provided as a liquid formulation, for example as one or two doses or as a freeze-dried (lyophilized) cake.

Component Formulations

In one aspect there is provided as a liquid formulation comprising:
a) a fusion protein as herein described,
b) optionally a liquid carrier such as water for injection, and
c) a stabilizing agent selected from glutathione, monothioglycerol cysteine, N-acetyl cysteine or mixtures thereof.

Liquid formulation in the above context can refer to a bulk product or a component of one or two doses.

The liquid formulation may, for example comprise a sugar such as saccharose, dextrose, mannitol or fructose, particularly saccharose. The amount of sugar may, for example be 1 to 10% by weight of the final formulation such as 4 to 5% w/w, such as 4% w/w.

The liquid formulation may, for example comprise arginine. Suitable amounts of arginine per dose are in the range 200 to 400 mM such as 300-375 mM, particularly to provide 300 mM in each final dose.

The liquid formulation may also comprise a chelating agent, for example citric acid trisodium salt, malic acid sodium salt, dextrose, L-methionine or EDTA disodium (ethylene diamine tretracetic acid), for example in the range 0.5 to 2 mM per dose such as 1 to 1.25 mM, particularly to provide 1 mM per final dose.

The liquid formulation may also comprise a non-ionic surfactant for example Tween such as Tween 80. Suitable amounts are in the range 0.005 to about 0.05% w/v such as 0.012 to 0.015% w/v, particularly 0.012% w/v in the final dose.

The Tween is used as a solubilising agent. However, it is thought that the Tween may contain residual peroxide that catalyses aggregation and/or degradation of the antigen. Advantageously use of an antioxidant according to the invention is thought to quench this reaction.

The liquid formulation may also comprise phosphate (PO₄) such as sodium phosphate, for example between 1 and 50 mM for example 10 mM such as 4 or 5 mM such as 4 mM in the final dose.

The liquid formulations of the invention may also include trace amounts of other components, for example which may be residual from the manufacturing process, for example tris HCL.

Thus in one aspect there is provided a final bulk or component for a HIV vaccine comprising:
a) an immunogenic fusion protein comprising Nef or an immunogenic fragment or derivative thereof, and p17 Gag and/or p24 Gag or immunogenic fragments or derivatives thereof, wherein when both p17 and p24 Gag are present there is at least one HIV antigen or immunogenic fragment between them,
b) a stabilising agent which is an antioxidant containing a thiol functional group, for example selected from the group consisting of glutathione, monothioglycerol, cysteine, N-acetyl cysteine or mixtures thereof,
c) 1% w/v or less of a non-ionic surfactant,
d) 200 to 450 mM of arginine
e) 0.5 to 2.0 mM of a chelating agent, and
f) 1 to 50 mM of a buffer.

In one aspect the component or a final formulation according to the invention further comprises a preservative, for example thiomersal. This may be a requirement when two or more doses, such as 10 doses, are supplied together.

A thiol functional group in the context of the present invention is intended to refer to at least one —SH group in the relevant molecule.

Final bulk in the context of this specification relates to purified antigen, carrier and other excipients but generally will not including adjuvant components/excipients. The bulk aspect refers to the presence of more than two doses in a given container. Thus final bulk is the formulation containing antigen and all excipients but minus adjuvant and before division into individual doses.

Purified bulk is intended to refer to antigen an minimal excipients, for example purified antigen suspended in phosphate saline buffer.

Component for a HIV vaccine herein refers to one or two doses of antigen and all excipient components, excluding adjuvant excipients.

In one aspect of the invention the Purified Bulk is produced in the following buffer: Tris 10 mM, Arginine 400 mM (100, 200 or 300 Mm), sodium sulfite 10 mM, EDTA 1 mM, residual Tween 80 at pH 8.5.

The invention also extends to a liquid formulation comprising sulfite but further comprising an antioxidant with at least one thiol group, as employed in the present invention. The sulfite may, for example be present at levels of 1% or below, such as 0.5% or below, particularly 0.1% or below, especially 0.05% or below (w/w or w/v)

In one embodiment any residual sulfite stabilizing agent in the bulk purified antigen (the latter being a component in the final bulk) is removed to provide a final bulk without any residual sulfite. In this aspect the final bulk will have a sulfite content less than 0.05% such as less than 0.01%, particularly zero.

This bulk may be freeze-dried (lyophilized) to provide cakes for reconstitution with an adjuvant.

In one embodiment a human dose 500 μl for cakes reconstituted with 625 μl of adjuvant comprises:

| | |
|---|---|
| F4 | 10-30-90 μg |
| saccharose | 4% |
| Arginine | 300 mM |
| N-acetyl cysteine | 0.5% w/v |
| EDTA disodium | 1 mM |
| Tween 80 | 0.012% w/v |
| PO4 | 4 mM |
| Tris-HCl | Residual |
| pH | 6.1 +/− 0.2 (when reconstituted with adjuvant but if |
| | reconstituted with water for injection then the pH is |
| | about 7.5) |

The pH of the final liquid formulation before the addition of liquid adjuvant formulation may be pH 6.50-pH 8.5 such as about pH 7.5. such as 7.5+/−0.1

In another embodiment the final bulk is divided into individual vials containing one or two doses of liquid formulation. This liquid formulation may be reconstituted with adjuvant as described above or can be freeze-dried for later reconstitution with for example adjuvant or water for injection.

Thus the liquid formulation may comprise said antigen, stabilizing agent and a liquid carrier, such as water for injection, but generally will contain all excipients, for example as for final bulk, excluding adjuvant excipients/components.

The pH of the reconstituted formulation according to the invention before the addition of liquid adjuvant formulation may be, for example pH 6.00 to pH 7.00 such as about pH 6.1.

In one embodiment there is provided a final liquid antigen formulation. Final liquid antigen formulation in the context of the present specification is intended to refer to less than 10 doses such as one or two doses of antigen with all the excipient other than adjuvant components.

Thus final liquid antigen formulation and component for a HIV vaccine are used interchangeably herein.

Vaccine (or final vaccine formulation) in the context of this specification is a formulation suitable for injection into a human patient and may for example be a final liquid formulation plus adjuvant components or lyophilized antigen reconstituted with adjuvant, as appropriate.

In one embodiment there is provided a final vaccine formulation according to the invention. Final formulation herein refers to a formulation containing all the necessary vaccine components including adjuvant components.

It may be advantageous to provide the vaccine formulation as separate components, for example in two liquid formulations (liquid antigen formulation and liquid adjuvant formulation) in separate vials because the antigen may have a longer shelf life in this form, in comparison to a form where a vaccine formulation is provided with all the components present (including adjuvant components).

Liquid component including for example liquid adjuvant formulation may require storage at about 4° C.

In one embodiment the antigen and stabilizing agent according to the invention are lyophilized. Adequate lyophilization may require the presence of a sugar or other excipients, for example as listed herein such as saccharose. In this embodiment one or more of the final bulk formulations described herein may be lyophilized with a stabilizing agent employed in the invention, for example N-acetyl cysteine, cysteine, monothioglycerol or mixtures thereof, such as N-acetyl cysteine, cysteine or monothioglycerol.

Providing a lyophilized product may have the advantage of providing a component that is very stable for long periods of time, for example in comparison to a final liquid formulation. A lyophilized product as described herein is more stable than a corresponding lyophilized product absent a stabilizing agent particularly when the antigen is present in a “high” concentration/dose, for example doses over 50 ug such as 60, 70, 80, 90 or 100 ug or more.

During lyophilization the effective amount of a component in the formulation may be reduced, which must be taken into account when preparing the product. Thus when the term final dose is used herein this refers to a vaccine formulation including a reconstituted dose suitable or ready for administration to a patient, thereby taking into account any loses as a result of lyophization.

The invention also extends to a pre-filled syringe containing a final liquid formulation or
a) a liquid component comprising the antigen and a stabilizing agent according to the invention, or
b) a liquid adjuvant formulation.

When the syringe contains a liquid component comprising an antigen and stabilizing agent then adjuvant may be drawn into the syringe to provide a final formulation for administration to a patient.

The pre-filled syringe containing antigen and a vial containing adjuvant may be provided as a kit.

Alternatively, where the adjuvant as pre-filled into the syringe then liquid antigen may be drawn into the syringe to provide a final formulation for administration to the patient.

The pre-filled syringe containing the adjuvant and a vial containing liquid antigen or lyophilized antigen may be provided as a kit. In this latter instance (ie when the antigen is lyophilized) the adjuvant in the syringe can be used to reconstitute the antigen in the vial and this vaccine formulation can then be drawn back into the syringe as required and administered to a patient.

Alternatively, a kit may be provided with a vial pre-filled with adjuvant and a separate vial of lyophilized antigen or liquid antigen according to the invention.

The invention also extends to a method or process of lyophilizing a component or composition according to the invention. The invention also extends to a process for forming a vaccine by combining;
a) a liquid antigen component according to the invention and a liquid adjuvant formulation to provide a final vaccine (such as one final dose of vaccine or two final doses of vaccine); or
b) a lyophilized antigen formulation according to the invention and a liquid adjuvant formulation to provide a final vaccine.

In one embodiment unsiliconised glass vials are employed to store the final bulk.

In one embodiment 3 mL siliconised glass vials are employed for containing the antigen components according to the invention or final vaccine formulation.

In one aspect of the invention the vials employed to store the component formulation according to the invention or vaccine formulation according to the invention is amber to protect said formulation from light.

Expression

Polynucleotides may be used to express the encoded polypeptides in a selected expression system. At least one of the HIV antigens, for example the RT, may be encoded by a codon optimized sequence in the polynucleotide, that is to say the sequence has been optimized for expression in a selected recombinant expression system such as E. coli.

A p51 RT polypeptide or derivative thereof or a polynucleotide encoding it, optionally codon-optimized for expression in a suitable expression system, particularly a prokaryotic system such as E. coli may be used.

The p51 RT polypeptide or polynucleotide may be used alone, or in combination with a polypeptide or polynucleotide construct

Processes

A polypeptide as described herein may, for example be purified by a process which comprises:
i) Providing a composition comprising the unpurified polypeptide;
ii) Subjecting the composition to at least two chromatographic steps;
iii) Optionally carboxyamidating the polypeptide;
iv) Performing a buffer exchange step to provide the protein in a suitable buffer for a pharmaceutical formulation.

The carboxyamidation may be performed between the two chromatographic steps. The carboxyamidation step may be performed using iodoacetimide.

In one process no more than two chromatographic steps, are employed.

In one aspect the invention provides a method for the preparation of a final bulk or a vaccine component as shown in the following flow diagram

Compositions/Methods of Treatment

Stabilized fusion proteins according to the invention may co-administered and/or co-formulated with:
one or more additional HIV polypeptides and/or HIV fusion proteins
polynucleotides encoding fusion proteins employed in the invention, and/or
viral vectors such as adenoviral vectors encoding one or more HIV antigens, particularly as described herein.

The polynucleotides may be present within any of a variety of delivery systems known to those of ordinary skill in the art, including nucleic acid expression systems such as plasmid DNA, bacterial and viral expression systems. Numerous gene delivery techniques are well known in the art, such as those described by Rolland, Crit. Rev. Therap. Drug Carrier Systems 15:143-198, 1998 and references cited therein. Appropriate nucleic acid expression systems contain the necessary DNA sequences for expression in the patient (such as a suitable promoter and terminating signal).

When the expression system is a recombinant live microorganism, such as a virus or bacterium, the gene of interest can be inserted into the genome of the live recombinant virus or bacterium. Inoculation and in vivo infection with this live vector will lead to in vivo expression of the antigen and induction of immune responses. Viruses and bacteria used for this purpose are for instance: poxviruses (e.g; vaccinia, fowlpox, canarypox, modified poxviruses e.g. Modified Virus Ankara (MVA)), alphaviruses (Sindbis virus, Semliki Forest Virus, Venezuelian Equine Encephalitis Virus), flaviviruses (yellow fever virus, Dengue virus, Japanese encephalitis virus), adenoviruses, adeno-associated virus, picornaviruses (poliovirus, rhinovirus), herpesviruses (varicella zoster virus, etc), morbilliviruses (e.g. measles such as Schwartz strain or a strain derived therefrom), Listeria, Salmonella, Shigella, Neisseria, BCG. These viruses and bacteria can be virulent, or attenuated in various ways in order to obtain live vaccines.

Adenovirus for use as a live vector include for example Ad5 or Ad35 or a non-human originating adenovirus such as a non-human primate adenovirus such as a simian adenovirus. Generally the vectors are replication defective. Typically these viruses contain an E1 deletion and can be grown on cell lines that are transformed with an E1 gene. Suitable simian adenoviruses are viruses isolated from chimpanzee. In particular C68 (also known as Pan 9) (See U.S. Pat. No. 6,083,716) and Pan 5, 6 and Pan 7 (WO03/046124) are preferred for use in the present invention. These vectors can be manipulated to insert a heterologous polynucleotide such that the polypeptides maybe expressed in vivo. The use, formulation and manufacture of such recombinant adenoviral vectors is described in detail in WO 03/046142.

The compositions of the invention may also include other HIV antigens in admixture such as gp120 polypeptides, NefTat fusion proteins, for example as described in WO 99/16884. Preparation of NefTat fusion proteins and also gp120 polypeptides/proteins is described in WO 01/54719.

In one embodiment gp120 polypeptide/protein is in admixture in the formulation according to the invention.

Vaccines employing components according to the invention may be used for prophylactic and/or therapeutic immunization against/for HIV and/or AIDS, particularly HIV.

The invention further provides the use of any aspect as described herein, in the manufacture of a vaccine for prophylactic and/or therapeutic immunization against/for HIV and/or AIDS, particularly HIV.

Vaccine preparation is generally described in New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

The amount of protein in the vaccine dose is selected as an amount which induces an appropriate immune response or immunoprotective response without significant, adverse side effects in typical vaccinees. Such amount will vary depending upon which specific immunogen is employed and the vaccination regimen that is selected. Generally, it is expected that each dose will comprise 1-1000 μg of each protein, for example 2-200 μg, such as 3-100 μg, particularly 10, 20, 30, 40, 50, 60, 70, 80 or 90 μg, especially 10, 30 or 90 μg of the polypeptide fusion (also referred to herein as fusion protein).

If gp120 is employed in admixture in the formulation the amount per dose will, for example be less than 100 μg such as 50 μg or less particularly 25, 20, 10, 5 μg.

An optimal amount for a particular vaccine can be ascertained by standard studies involving observation of antibody titres and other immune responses in subjects.

Following an initial vaccination, subjects may receive a boost in about 4, 5, 6, 7, 8, 9, 10, 11, 12, 16 or 24 weeks, and a subsequent second booster in a further 4, 5, 6, 7, 8, 9, 10, 11 or 12, 16, 20, 24, 28, 32, 36, 40, 44, 48 or 50 weeks.

Alternatively subjects may receive a boost in about 4, 5, 6, 7, 8, 9, 10, 11, 12, 16 or 24 weeks, and a subsequent second booster in a further 4, 8, 12, 16, 20, 24, 28, 32, 36, 40, 44, 48, or 52 weeks.

The final vaccine formulation of fusion protein suitable for administration will comprise an adjuvant.

Adjuvants are described in general in Vaccine Design—the Subunit and Adjuvant Approach, edited by Powell and Newman, Plenum Press, New York, 1995.

Suitable adjuvants include an aluminium salt such as aluminium hydroxide or aluminium phosphate, but may also be a salt of calcium, iron or zinc, or may be an insoluble suspension of acylated tyrosine, or acylated sugars, cationically or anionically derivatised polysaccharides, or polyphosphazenes.

In the formulation of the invention a suitable adjuvant composition is one which induces a preferential Th1 response.

The mammalian immune response has two key components: the humoral response and the cell-mediated response.

The humoral response involves the generation of circulating antibodies which will bind to the antigen to which they are specific, thereby neutralising the antigen and favouring its subsequent clearance by a process involving other cells that are either cytotoxic or phagocytic. B-cells are responsible for generating antibodies (plasma B cells), as well as holding immunological humoral memory (memory B-cells), i.e. the ability to recognise an antigen some years after first exposure to it eg through vaccination.

The cell mediated response involves the interplay of numerous different types of cells, among which are the T cells. T-cells are divided into a number of different subsets, mainly the CD4+ and CD8+ T cells.

Antigen-presenting cells (APC) such as macrophages and dendritic cells act as sentinels of the immune system, screening the body for foreign antigens. When extracellular foreign antigens are detected by APC, these antigens are phagocytosed (engulfed) inside the APC where they will be processed into smaller peptides. These peptides are subsequently presented on major histocompatibility complex class II (MHC II) molecules at the surface of the APC where they can be recognised by antigen-specific T lymphocytes expressing the CD4 surface molecules (CD4+ T cells).

T helper CD4+ T cells provide help to activate B cells to produce and release antibodies. T helper CD4+ T cells can also participate to the activation of antigen-specific CD8+ T cells.

CD8+ T cells recognize the peptide to which they are specific when it is presented on the surface of a host cell by major histocompatibility class I (MHC I) molecules in the presence of appropriate costimulatory signals. In order to be presented on MHC I molecules, a foreign antigen need to directly access the inside of the cell (the cytosol or nucleus) such as it is the case when a virus or intracellular bacteria directly penetrate a host cell or after DNA vaccination. Inside the cell, the antigen is processed into small peptides that will be loaded onto MHC I molecules that are redirected to the surface of the cell. Upon activation CD8+ T cells secrete an array of cytokines such as interferon gamma that activates macrophages and other cells. In particular, a subset of these CD8+ T cells secretes lytic and cytotoxic molecules (e.g. granzyme, perforin) upon activation. Such CD8+ T cells are referred to as cytotoxic T cells.

More recently, an alternative pathway of antigen presentation involving the loading of extracellular antigens or fragments thereof onto MHCI complexes has been described and called “cross-presentation”.

Among the CD4+ T cells, the T helper 1 (Th1) and the T helper 2 (Th2) subsets can be defined by the type of response they generate following antigen recognition. Upon recognition of a peptide-MHC II complex, Th1 CD4+ T cells secrete interleukins and cytokines such as interferon gamma, IL-2 and TNF-alpha. In contrast, Th2 CD4+ T cells generally secrete interleukins such as IL-4, IL-5 or IL-13.

It is known that certain vaccine adjuvants are particularly suited to the stimulation of either Th1 or Th2 -type cytokine responses. Traditionally the best indicators of the Th1:Th2 balance of the immune response after a vaccination or infection includes direct measurement of the production of Th1 or Th2 cytokines by T lymphocytes in vitro after restimulation with antigen, and/or the measurement of the IgG1:IgG2a ratio of antigen specific antibody responses.

Thus, a Th1-type adjuvant is one which stimulates isolated T-cell populations to produce high levels of Th1-type cytokines when re-stimulated with antigen in vitro, and induces antigen specific immunoglobulin responses associated with Th1-type isotype.

Preferred Th1-type immunostimulants which may be formulated to produce adjuvants suitable for use in the present invention include and are not restricted to the following.

Monophosphoryl lipid A, in particular 3-de-O-acylated monophosphoryl lipid A (3D-MPL), is a preferred Th1 -type immunostimulant for use in the invention. 3D-MPL is a well known adjuvant manufactured by Ribi Immunochem, Montana. Chemically it is often supplied as a mixture of 3-de-O-acylated monophosphoryl lipid A with either 4, 5, or 6 acylated chains. It can be purified and prepared by the methods taught in GB 2122204B, which reference also discloses the preparation of diphosphoryl lipid A, and 3-O-deacylated variants thereof. Other purified and synthetic lipopolysaccharides have been described (U.S. Pat. No. 6,005,099 and EP 0 729 473 B1; Hilgers et al., 1986, Int. Arch. Allergy. Immunol., 79(4):392-6; Hilgers et al., 1987, Immunology, 60(1):141-6; and EP 0 549 074 B1). A preferred form of 3D-MPL is in the form of a particulate formulation having a small particle size less than 0.2 μm in diameter, and its method of manufacture is disclosed in EP 0 689 454.

Saponins are also preferred Th1 immunostimulants in accordance with the invention. Saponins are well known adjuvants and are taught in: Lacaille-Dubois, M and Wagner H. (1996. A review of the biological and pharmacological activities of saponins Phytomedicine vol 2 pp 363-386). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Also described in these references is the use of QS7 (a non-haemolytic fraction of Quil-A) which acts as a potent adjuvant for systemic vaccines. Use of QS21 is further described in Kensil et al. (1991. J. Immunology vol 146, 431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711. One such system is known as an ISCOM and may contain one or more saponins.

Another suitable immunostimulant is an immunostimulatory oligonucleotide containing unmethylated CpG dinucleotides (“CpG”). CpG is an abbreviation for cytosine-guanosine dinucleotide motifs present in DNA. CpG is known in the art as being an adjuvant when administered by both systemic and mucosal routes (WO 96/02555, EP 468520, Davis et al., J. Immunol, 1998, 160(2):870-876; McCluskie and Davis, J. Immunol., 1998, 161(9):4463-6). Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, Nature 374, p546 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the CG motif is not methylated, but other unmethylated CpG sequences are known to be immunostimulatory and may be used in the present invention.

In some instances combinations of the six nucleotides a palindromic sequence are present. Several of these motifs, either as repeats of one motif or a combination of different motifs, can be present in the same oligonucleotide. The presence of one or more of these immunostimulatory sequences containing oligonucleotides can activate various immune subsets, including natural killer cells (which produce interferon γ and have cytolytic activity) and macrophages (Wooldrige et al Vol 89 (no. 8), 1977). Other unmethylated CpG containing sequences not having this consensus sequence have also now been shown to be immunomodulatory.

It is also hypothesized by the inventors that in fact these “CpG” containing sequences are also susceptible to oxidation and the addition of a thiol containing reducing group as employed in the present invention is thought to have the further benefit of reducing or eliminating this undesirable oxidation.

CpG when formulated into vaccines, is generally administered in free solution together with free antigen (WO 96/02555; McCluskie and Davis, supra) or covalently conjugated to an antigen (WO 98/16247), or formulated with a carrier such as aluminium hydroxide ((Hepatitis surface antigen) Davis et al. supra ; Brazolot-Millan et al., Proc. Natl. Acad. Sci., USA, 1998, 95(26), 15553-8).

Such immunostimulants as described above may be formulated together with carriers, such as for example liposomes, oil in water emulsions, and or metallic salts, including aluminium salts (such as aluminium hydroxide). For example, 3D-MPL may be formulated with aluminium hydroxide (EP 0 689 454) or oil in water emulsions (WO 95/17210); QS21 may be advantageously formulated with cholesterol containing liposomes (WO 96/33739), oil in water emulsions (WO 95/17210) or alum (WO 98/15287); CpG may be formulated with alum (Davis et al. supra; Brazolot-Millan supra) or with other cationic carriers.

Combinations of immunostimulants are also preferred, in particular a combination of a monophosphoryl lipid A and a saponin derivative (WO 94/00153; WO 95/17210; WO 96/33739; WO 98/56414; WO 99/12565; WO 99/11241), more particularly the combination of QS21 and 3D-MPL as disclosed in WO 94/00153. Alternatively, a combination of CpG plus a saponin such as QS21 also forms a potent adjuvant for use in the present invention. Alternatively the saponin may be formulated in a liposome or in an ISCOM and combined with an immunostimulatory oligonucleotide.

An enhanced system involves the combination of a monophosphoryl lipid A and a saponin derivative particularly the combination of QS21 and 3D-MPL as disclosed in WO 94/00153, or a less reactogenic composition where the QS21 is quenched in cholesterol containing liposomes (DQ) as disclosed in WO 96/33739. This combination may additionally comprise an immunostimulatory oligonucleotide.

A particularly potent adjuvant formulation involving QS21, 3D-MPL & tocopherol in an oil in water emulsion is described in WO 95/17210 and is another suitable formulation for use in the invention.

Particularly suitable adjuvant combinations for use in the formulations according to the invention are as follows:

i) 3D-MPL+QS21 in a liposomal formulation

ii) 3D-MPL+QS21 in an oil in water emulsion

iii) 3D-MPL+QS21+CpG in a liposomal formulation, and

iv) 3D-MPL+QS21+CpG in an oil in water emulsion

In a further aspect of the present invention there is provided a method of manufacture of a vaccine formulation as herein described, wherein the method comprises admixing a polypeptide according to the invention with a suitable adjuvant.

Administration of the pharmaceutical composition may take the form of one or of more than one individual dose, for example as repeat doses of the same polypeptide containing composition, or in a heterologous “prime-boost” vaccination regime. A heterologous prime-boost regime uses administration of different forms of vaccine in the prime and the boost, each of which may itself include two or more administrations. The priming composition and the boosting composition will have at least one antigen in common, although it is not necessarily an identical form of the antigen, it may be a different form of the same antigen.

Prime boost immunisations according to the invention may be performed with a combination of protein and DNA-based or viral vector formulations. Such a strategy is considered to be effective in inducing broad immune responses. Adjuvanted protein vaccines induce mainly antibodies and T helper immune responses, while delivery of DNA as a plasmid or a live vector induces strong cytotoxic T lymphocyte (CTL) responses. Thus, the combination of protein and DNA or viral vector vaccination will provide for a wide variety of immune responses. This is particularly relevant in the context of HIV, since neutralising antibodies, CD4+ T cells and/or CTL are thought to be important for the immune defense against HIV.

In accordance with the invention a schedule for vaccination may comprise the sequential (“prime-boost”) administration of polypeptide antigens according to the invention and DNA encoding the polypeptides. The DNA may be delivered as naked DNA such as plasmid DNA or in the form of a recombinant live vector, e.g. a poxvirus vector, an adenovirus vector, or any other suitable live vector. Protein antigens may be injected once or several times followed by one or more DNA or viral vector administrations, or DNA or viral vector may be used first for one or more administrations followed by one or more protein immunisations.

A particular example of prime-boost immunisation according to the invention involves priming with DNA a recombinant live vector such as a modified poxvirus vector, for example Modified Virus Ankara (MVA) or an alphavirus, for example Venezuelian Equine Encephalitis Virus, or an adenovirus vector, followed by boosting with a protein, such as an adjuvanted protein.

Both the priming composition and the boosting composition may be delivered in more than one dose. Furthermore the initial priming and boosting doses may be followed up with further doses which may be alternated to result in e.g. a DNA plasmid or viral vector prime/protein boost/further DNA plasmid or viral vector dose/further protein dose. An alternative prime boost regime may for example include priming with one or two doses of protein, with one or two subsequent boosts with DNA or viral vector.

By codon optimisation it is meant that the polynucleotide sequence, is optimised to resemble the codon usage of genes in the desired expression system, for example a prokaryotic system such as E. coli. In particular, the codon usage in the sequence is optimised to resemble that of highly expressed E. coli genes.

The purpose of codon optimizing for expression in a recombinant system according to the invention is twofold: to improve expression levels of the recombinant product and to render expression products more homogeneous (obtain a more homogeneous expression pattern). Improved homogeneity means that there are fewer irrelevant expression products such as truncates. Codon usage adaptation to E. coli expression can also eliminate the putative “frame-shift” sequences as well as premature termination and/or internal initiation sites.

The DNA code has 4 letters (A, T, C and G) and uses these to spell three letter “codons” which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons.

Where more than one codon is available to code for a given amino acid, it has been observed that the codon usage patterns of organisms are highly non-random. Different species show a different bias in their codon selection and, furthermore, utilisation of codons may be markedly different in a single species between genes which are expressed at high and low levels. This bias is different in viruses, plants, bacteria and mammalian cells, and some species show a stronger bias away from a random codon selection than others. For example, humans and other mammals are less strongly biased than certain bacteria or viruses. For these reasons, there is a significant probability that a viral gene from a mammalian virus expressed in E. coli, or a foreign or recombinant gene expressed in mammalian cells will have an inappropriate distribution of codons for efficient expression. It is believed that the presence in a heterologous DNA sequence of clusters of codons or an abundance of codons which are rarely observed in the host in which expression is to occur, is predictive of low heterologous expression levels in that host.

In the polynucleotides of the present invention, the codon usage pattern may thus be altered from that typical of human immunodeficiency viruses to more closely represent the codon bias of the target organism, e.g. E. coli.

There are a variety of publicly available programs useful for codon optimization, for example “CalcGene” (Hale and Thompson, Protein Expression and Purification 12: 185-189 (1998).

The invention also extends to use of glutathione, monothioglycerol, cysteine and N-acetyl cysteine or mixtures thereof (particularly monothioglycerol, cysteine or N-acetyl cysteine) to stabilise a component for a HIV vaccine, for example comprising an immunogenic fusion protein comprising Nef or an immunogenic fragment or derivative thereof, and p17 Gag and/or p24 Gag or immunogenic fragments or derivatives thereof, wherein when both p17 and p24 Gag are present there is at least one HIV antigen or immunogenic fragment between them, particularly F4.

In an alternative or additional aspect the invention provides a protein described herein, such as F4 protein in an inert environment , for example in a container wherein the oxygen has been removed and/or the protein is protected from light. This also seems to be able to minimize or eliminate the aggregation and/or degradation of the protein. The protein may, for example be stored under nitrogen and/or stored in an amber vial.

Comprising in the context of this specification is intended to be inclusive, that is to say the embodiment includes the relevant elements, without the exclusion of other elements.

The invention also extends to separate embodiments consisting or consisting essentially of the elements described herein as aspects/embodiments comprising said elements and vice versa.

Description in the background section of this document is for the purpose of putting the invention into context. It is not to be taken as an admission that the information is known or is common general knowledge.

The examples below are shown to illustrate the methodology, which may be employed to prepare particles of the invention.

### EXAMPLES

### Example 1

### Construction and Expression of HIV-1 p24-RT-Nef-p17 Fusion F4 and F4 Codon Optimized (co)

1. F4 Non-Codon-Optimised

HIV-1 gag p24 (capsid protein) and p17 (matrix protein), the reverse transcriptase and Nef proteins were expressed in E. coli B834 strain (B834 (DE3) is a methionine auxotroph parent of BL21 (DE3)), under the control of the bacteriophage T7 promoter (pET expression system).

They were expressed as a single fusion protein containing the complete sequence of the four proteins. Mature p24 coding sequence comes from HIV-1 BH10 molecular clone, mature p17 sequence and RT gene from HXB2 and Nef gene from the BRU isolate.

After induction, recombinant cells expressed significant levels of the p24-RT-Nef-p17 fusion that amounted to 10% of total protein.

When cells were grown and induced at 22° C., the p24-RT-Nef-p17 fusion protein was confined mainly to the soluble fraction of bacterial lysates (even after freezing/thawing). When grown at 30° C., around 30% of the recombinant protein was associated with the insoluble fraction.

The fusion protein p24-RT-Nef-p17 is made up of 1136 amino acids with a molecular mass of approximately 129 kDa. The full-length protein migrates to about 130 kDa on SDS gels. The protein has a theoretical isoelectric point (pI) of 7.96 based on its amino acid sequence, confirmed by 2D-gel electrophoresis.

Details of the Recombinant Plasmid:

name: pRIT15436 (or lab name pET28b/p24-RT-Nef-p17)
host vector: pET28b
replicon: colE1

selection: kanamycin

promoter: T7

insert: p24-RT-Nef-p17 fusion gene.

Details of the Recombinant Protein:

p24-RT-Nef-p17 fusion protein : 1136 amino acids.

N-term-p24: 232a.a.-hinge:2a.a.-RT: 562a.a.-hinge:2a.a.-Nef: 206a.a.-P17: 132a.a.-C-term

Nucleotide and Amino-Acid Sequences:

Nucleotide Sequence

[SEQ ID NO: 1]
atggttatcgtgcagaacatccaggggcaaatggtacatcaggccat
atcacctagaactttaaatgcatgggtaaaagtagtagaagagaagg
ctttcagcccagaagtaatacccatgttttcagcattatcagaagga
gccaccccacaagatttaaacaccatgctaaacacagtggggggaca
tcaagcagccatgcaaatgttaaaagagaccatcaatgaggaagctg
cagaatgggatagagtacatccagtgcatgcagggcctattgcacca
ggccagatgagagaaccaaggggaagtgacatagcaggaactactag
tacccttcaggaacaaataggatggatgacaaataatccacctatcc
cagtaggagaaatttataaaagatggataatcctgggattaaataaa
atagtaagaatgtatagccctaccagcattctggacataagacaagg
accaaaagaaccttttagagactatgtagaccggttctataaaactc
taagagccgagcaagcttcacaggaggtaaaaaattggatgacagaa
accttgttggtccaaaatgcgaacccagattgtaagactattttaaa
agcattgggaccagcggctacactagaagaaatgatgacagcatgtc
agggagtaggaggacccggccataaggcaagagttttg [symbol] ggc
cccattagccctattgagactgtgtcagtaaaattaaagccaggaat
ggatggcccaaaagttaaacaatggccattgacagaagaaaaaataa
aagcattagtagaaatttgtacagagatggaaaaggaagggaaaatt
tcaaaaattgggcctgaaaatccatacaatactccagtatttgccat
aaagaaaaaagacagtactaaatggagaaaattagtagatttcagag
aacttaataagagaactcaagacttctgggaagttcaattaggaata
ccacatcccgcagggttaaaaaagaaaaaatcagtaacagtactgga
tgtgggtgatgcatatttttcagttcccttagatgaagacttcagga
aatatactgcatttaccatacctagtataaacaatgagacaccaggg
attagatatcagtacaatgtgcttccacagggatggaaaggatcacc
agcaatattccaaagtagcatgacaaaaatcttagagccttttagaa
aacaaaatccagacatagttatctatcaatacatggatgatttgtat
gtaggatctgacttagaaatagggcagcatagaacaaaaatagagga
gctgagacaacatctgttgaggtggggacttaccacaccagacaaaa
aacatcagaaagaacctccattccttaaaatgggttatgaactccat
cctgataaatggacagtacagcctatagtgctgccagaaaaagacag
ctggactgtcaatgacatacagaagttagtggggaaattgaattggg
caagtcagatttacccagggattaaagtaaggcaattatgtaaactc
cttagaggaaccaaagcactaacagaagtaataccactaacagaaga
agcagagctagaactggcagaaaacagagagattctaaaagaaccag
tacatggagtgtattatgacccatcaaaagacttaatagcagaaata
cagaagcaggggcaaggccaatggacatatcaaatttatcaagagcc
atttaaaaatctgaaaacaggaaaatatgcaagaatgaggggtgccc
acactaatgatgtaaaacaattaacagaggcagtgcaaaaaataacc
acagaaagcatagtaatatggggaaagactcctaaatttaaactgcc
catacaaaaggaaacatgggaaacatggtggacagagtattggcaag
ccacctggattcctgagtgggagtttgttaatacccctcctttagtg
aaattatggtaccagttagagaaagaacccatagtaggagcagaaac
cttctatgtagatggggcagctaacagggagactaaattaggaaaag
caggatatgttactaatagaggaagacaaaaagttgtcaccctaact
gacacaacaaatcagaagactgagttacaagcaatttatctagcttt
gcaggattcgggattagaagtaaacatagtaacagactcacaatatg
cattaggaatcattcaagcacaaccagatcaaagtgaatcagagtta
gtcaatcaaataatagagcagttaataaaaaaggaaaaggtctatct
ggcatgggtaccagcacacaaaggaattggaggaaatgaacaagtag
ataaattagtcagtgctggaatcaggaaagtgcta [symbol] ggtggc
aagtggtcaaaaagtagtgtggttggatggcctactgtaagggaaag
aatgagacgagctgagccagcagcagatggggtgggagcagcatctc
gagacctggaaaaacatggagcaatcacaagtagcaatacagcagct
accaatgctgcttgtgcctggctagaagcacaagaggaggaggaggt
gggttttccagtcacacctcaggtacctttaagaccaatgact
tacaaggcagctgtagatcttagccactttttaaaagaaaagggggg
actggaagggctaattcactcccaacgaagacaagatatccttgatc
tgtggatctaccacacacaaggctacttccctgattggcagaactac
acaccagggccaggggtcagatatccactgacctttggatggtgcta
caagctagtaccagttgagccagataaggtagaagaggccaataaag
gagagaacaccagcttgttacaccctgtgagcctgcatggaatggat
gaccctgagagagaagtgttagagtggaggtttgacagccgcctagc
atttcatcacgtggcccgagagctgcatccggagtacttcaagaact
gc [symbol] atgggtgcgagagcgtcagtattaagcgggggagaatta
gatcgatgggaaaaaattcggttaaggccagggggaaagaaaaaata
taaattaaaacatatagtatgggcaagcagggagctagaacgattcg
cagttaatcctggcctgttagaaacatcagaaggctgtagacaaata
ctgggacagctacaaccatcccttcagacaggatcagaagaacttag
atcattatataatacagtagcaaccctctattgtgtgcatcaaagga
tagagataaaagacaccaaggaagctttagacaagatagaggaagag
caaaacaaaagtaagaaaaaagcacagcaagcagcagctgacacagg
acacagcaatcaggtcagccaaaattactaa
p24 sequence is in bold
Nef sequence is underlined
Boxes: nucleotides introduced by genetic construction

Amino-Acid sequence

| [SEQ ID NO: 2] | |
|---|---|
| MVIVQNIQGQMVHQAISPRTLNAWVKVVEEKAFSPEVIPMFSALSEGATP | 50 |
| QDLNTMLNTVGGHQAAMQMLKETINEEAAEWDRVHPVHAGPIAPGQMREP | 100 |
| RGSDIAGTTSTLQEQIGWMTNNPPIPVGEIYKRWIILGLNKIVRMYSPTS | 150 |
| ILDIRQGPKEPFRDYVDRFYKTLRAEQASQEVKNWMTETLLVQNANPDCK | 200 |
| TILKALGPAATLEEMMTACQGVGGPGHKARVL [symbol] GPISPIETVSVKLKPG | 250 |
| MDGPKVKQWPLTEEKIKALVEICTEMEKEGKISKIGPENPYNTPVFAIKK | 300 |
| KDSTKWRKLVDFRELNKRTQDFWEVQLGIPHPAGLKKKKSVTVLDVGDAY | 350 |
| FSVPLDEDFRKYTAFTIPSINNETPGIRYQYNVLPQGWKGSPAIFQSSMT | 400 |
| KILEPFRKQNPDIVIYQYMDDLYVGSDLEIGQHRTKIEELRQHLLRWGLT | 450 |
| TPDKKHQKEPPFL [symbol] MGYELHPDKWTVQPIVLPEKDSWTVNDIQKLVGKLN | 500 |
| WASQIYPGIKVRQLCKLLRGTKALTEVIPLTEEAELELAENREILKEPVH | 550 |
| GVYYDPSKDLIAEIQKQGQGQWTYQIYQEPFKNLKTGKYARMRGAHTNDV | 600 |
| KQLTEAVQKITTESIVIWGKTPKFKLPIQKETWETWWTEYWQATWIPEWE | 650 |
| FVNTPPLVKLWYQLEKEPIVGAETFYVDGAANRETKLGKAGYVTNRGRQK | 700 |
| VVTLTDTTNQKTELQAIYLALQDSGLEVNIVTDSQYALGIIQAQPDQSES | 750 |
| ELVNQIIEQLIKKEKVYLAWVPAHKGIGGNEQVDKLVSAGIRKV [symbol] MGGK | 800 |
| WSKSSVVGWPTVRERMRRAEPAADGVGAASRDLEKHGAITSSNTAATNAA | 850 |
| CAWLEAQEEEEVGFPVTPQVPLRPMTYKAAVDLSHFLKEKGGLEGLIHSQ | 900 |
| RRQDILDLWIYHTQGYFPDWQNYTPGPGVRYPLTFGWCYKLVPVEPDKVE | 950 |
| EANKGENTSLLHPVSLHGMDDPEREVLEWRFDSRLAFHHVARELHPEYFK | 1000 |
| NC [symbol] MGARASVLSGGELDRWEKIRLRPGGKKKYKLKHIVWASRELERFAV | 1050 |
| NPGLLETSEGCRQILGQLQPSLQTGSEELRSLYNTVATLYCVHQRIEIKD | 1100 |
| TKEALDKIEEEQNKSKKKAQQAAADTGHSNQVSQNY | 1136 |

P24 sequence: amino-acids 1-232 (in bold)
RT sequence: amino-acids 235-795
Nef sequence: amino-acids 798-1002
P17 sequence: amino-acids 1005-1136
Boxes: amino-acids introduced by genetic construction
K (Lysine): instead of Tryptophan (W). Mutation
introduced to remover enzyme activity.

Expression of the Recombinant Protein:

In pET plasmid, the target gene (p24-RT-Nef-p17) is under control of the strong bacteriophage T7 promoter. This promoter is not recognized by E. coli RNA polymerase and is dependent on a source of T7 RNA polymerase in the host cell. B834 (DE3) host cell contains a chromosomal copy of the T7 RNA polymerase gene under lacUV5 control and expression is induced by the addition of IPTG to the bacterial culture.

Pre-cultures were grown, in shake flasks, at 37° C. to mid-log phase (A620:0.6) and then stored at 4° C. overnight (to avoid stationary phase cultures). Cultures were grown in LBT medium supplemented with 1% glucose and 50 μg/ml kanamycin. Addition of glucose to the growth medium has the advantage to reduce the basal recombinant protein expression (avoiding cAMP mediated derepression of lacUV5 promoter)

Ten ml of cultures stored overnight at 4° C. were used to inoculate 200 ml of LBT medium (without glucose) containing kanamycin. Cultures were grown at 30° C. and 22° C. and when O.D.620 reached 0.6, IPTG was added (1 mM final). Cultures were incubated for further 3, 5 and 18 hours (overnight). Samples were collected before and after 3, 5 and 18 hours induction.

Extract preparation was as follows:

Cell pellets were suspended in breaking buffer* (at a theoretical O.D. of 10) and disrupted by four passages in French press (at 20.000 psi or 1250 bars). Crude extracts (T) were centrifuged at 20.000 g for 30 min to separate the soluble (S) and insoluble (P) fractions.

*Breaking buffer: 50 mM Tris-HCL pH 8.0, 1 mM EDTA, 1 mM DTT+protease inhibitors cocktail (Complete/Boerhinger).

SDS-PAGE and Western Blot analysis:

Fractions corresponding to insoluble pellet (P), supernatant (S) and crude extract (T) were run on 10% reducing SDS-PAGE. p24-RT-Nef-p17recombinant was detected by Coomassie blue staining and on Western blot (WB).

Coomassie staining: p24-RT-Nef-p17 protein appears as:
one band at ±130 kDa (fitting with calculated MW)
MW theoretical: 128.970 Daltons
MW apparent: 130 kDa

Western blot analysis:
Reagents=Monoclonal antibody to RT (p66/p51)
   Purchased from ABI (Advanced Biotechnologies) dilution: 1/5000
   Alkaline phosphatase-conjugate anti-mouse antibody dilution: 1/7500

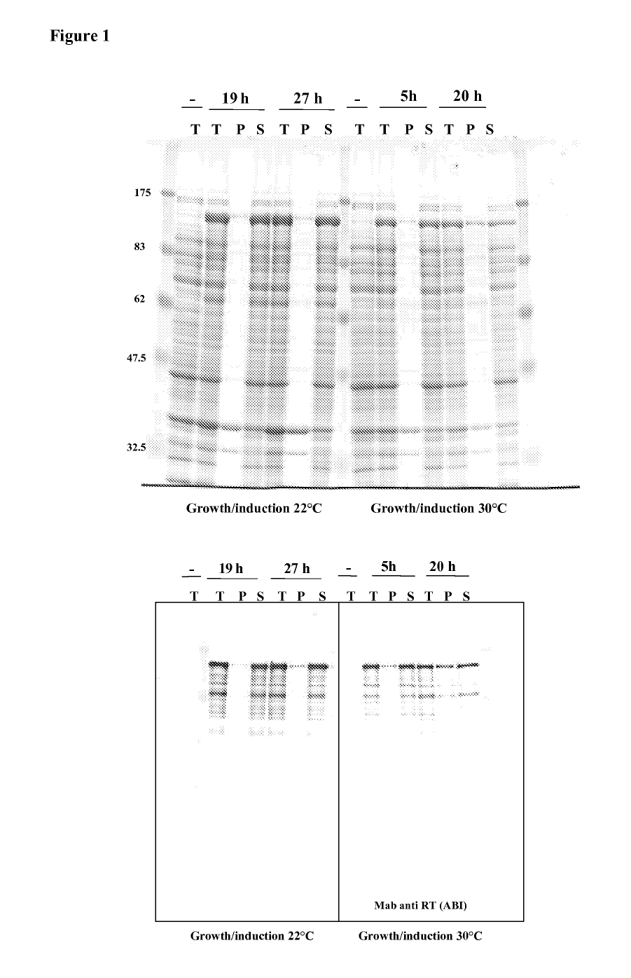

Expression level: Very strong p24-RT-Nef-p17 specific band after 20 h induction at 22° C., representing up to 10% of total protein (See FIG. 1).

Recombinant protein “solubility”:

“Fresh” cellular extracts (T,S,P fractions): With growth/induction at 22° C./20 h, almost all p24-RT-Nef-p17 fusion protein is recovered in the soluble fraction of cellular extract (FIG. 1). With growth/induction at 30° C./20 h, around 30% of p24-RT-Nef-p17 protein is associated with the insoluble fraction (FIG. 1).

“Freezing/thawing” (S2, P2 fractions):

Soluble (S1) fraction (20 h induction at 22° C.) conserved at −20° C. Thawed and centrifuged at 20.000 g/30 min : S2 and P2 (resuspended in 1/10 vol.)

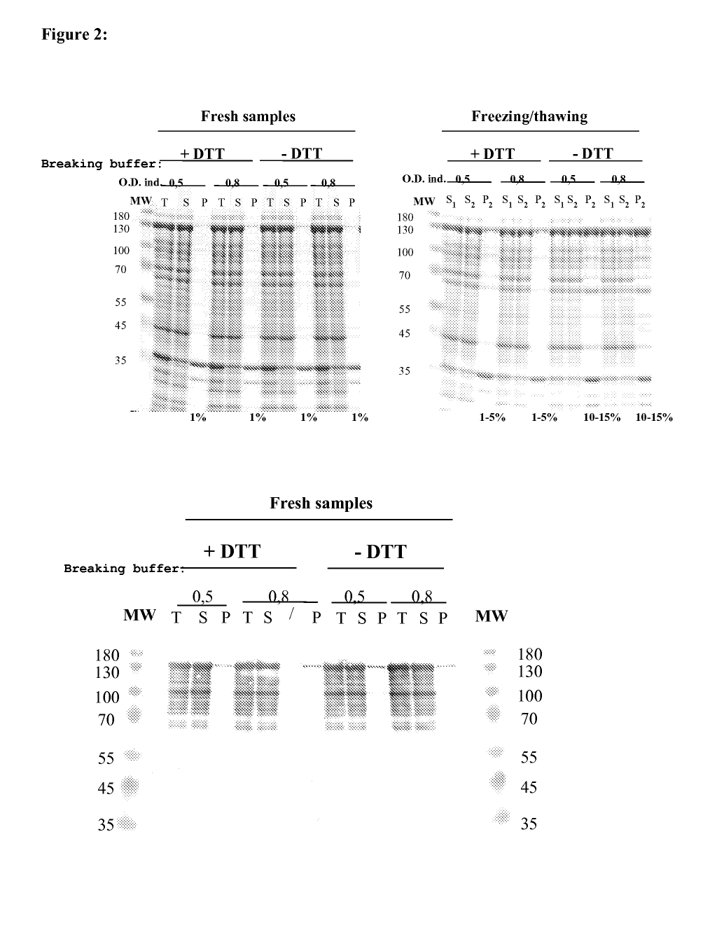

Breaking buffer with DTT : almost all p24-RT-Nef-p17 fusion protein still soluble (only 1-5% precipitated) (see FIG. 2)

Breaking buffer without DTT: 85-90% of p24-RT-Nef-p17 still soluble (FIG. 2)

### FIGURES

FIG. 1—Coomassie staining and western blot for p24-RT-Nef-p17 (F4) (10% SDS-PAGE-Reducing)

FIG. 2—p24-RT-Nef-p17 solubility assay detected by coomasie staining and western blot (Reducing gels—10% SDS-PAGE)

The cell growth and induction conditions and cellular extracts preparation for the examples which follow are as described in Example 1 unless other conditions are specified (e.g. temperature, composition of breaking buffer).

2. F4 Codon-Optimised

The following polynucleotide sequence is codon optimized such that the codon usage resembles the codon usage in a highly expressed gene in E. coli. The amino acid sequence is identical to that given above for F4 non-codon optimized.

Nucleotide Sequence for F4co:

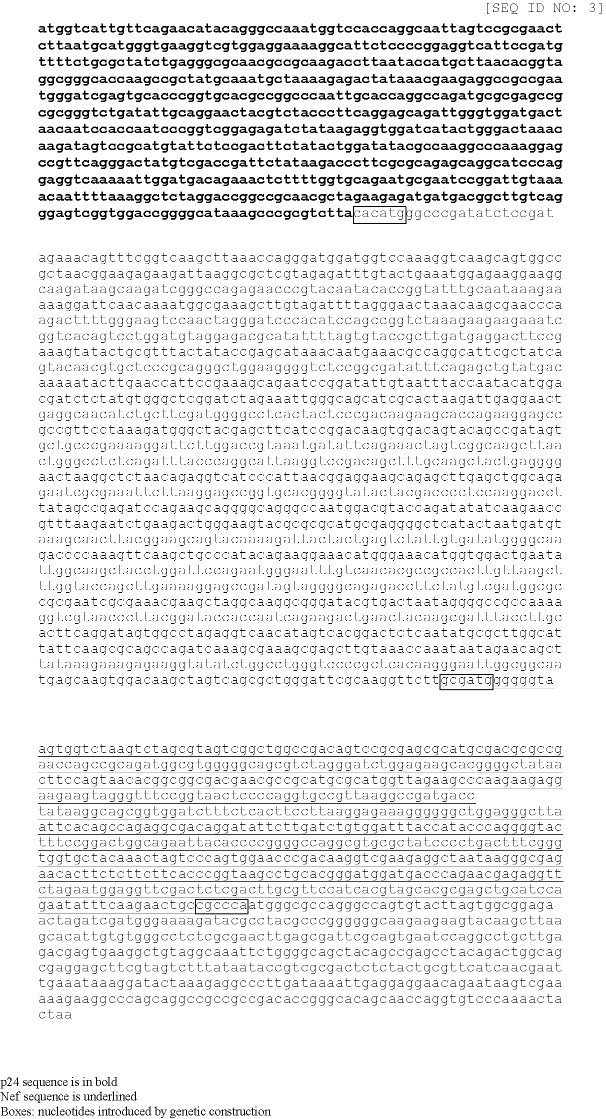

The procedures used in relation to F4 non-codon optimized were applied for the codon-optimised sequence.

Details of the Recombinant Plasmid:
name: pRIT15513 (lab name: pET28b/p24-RT-Nef -p17)
host vector: pET28b
replicon: colE1
selection: kanamycin
promoter: T7
insert: p24-RT-Nef-p17 fusion gene, codon-optimized

The F4 codon-optimised gene was expressed in E. coli BLR(DE3) cells, a recA⁻ derivative of B834(DE3) strain. RecA mutation prevents the putatitve production of lambda phages.

Pre-cultures were grown, in shake flasks, at 37° C. to mid-log phase (A₆₂₀:0.6) and then stored at 4° C. overnight (to avoid stationary phase cultures).

Cultures were grown in LBT medium supplemented with 1% glucose and 50 μg/ml kanamycin. Addition of glucose to the growth medium has the advantage to reduce the basal recombinant protein expression (avoiding cAMP mediated derepression of lacUV5 promoter).

Ten ml of cultures stored overnight at 4° C. were used to inoculate 200 ml of LBT medium (without glucose) containing kanamycin. Cultures were grown at 37° C. and when O.D.₆₂₀ reached 0.6, IPTG was added (1 mM final). Cultures were incubated for further 19 hours (overnight), at 22° C. Samples were collected before and 19 hours induction.

Extract preparation was as follows:

Cell pellets were resuspended in sample buffer (at a theoretical O.D. of 10), boiled and directly loaded on SDS-PAGE.

SDS-PAGE and Western Blot analysis:

Crude extracts samples were run on 10% reducing SDS-PAGE.

p24-RT-Nef -p17 recombinant protein is detected by Coomassie blue staining (FIG. 2) and on Western blot.
Coomassie staining: p24-RT-Nef-p17 protein appears as: one band at ±130 kDa (fitting with calculated MW)
   MW theoretical: 128.967 Daltons
   MW apparent: 130 kDa
Western blot analysis:
Reagents=Rabbit polyclonal anti RT (rabbit PO3L16) dilution: 1/10.000
   Rabbit polyclonal anti Nef-Tat (rabbit 388) dilution 1/10.000
   Alkaline phosphatase-conjugate anti-rabbit antibody. dilution: 1/7500

After induction at 22° C. over 19 hours, recombinant BLR(DE3) cells expressed the F4 fusion at a very high level ranging from 10-15% of total protein.

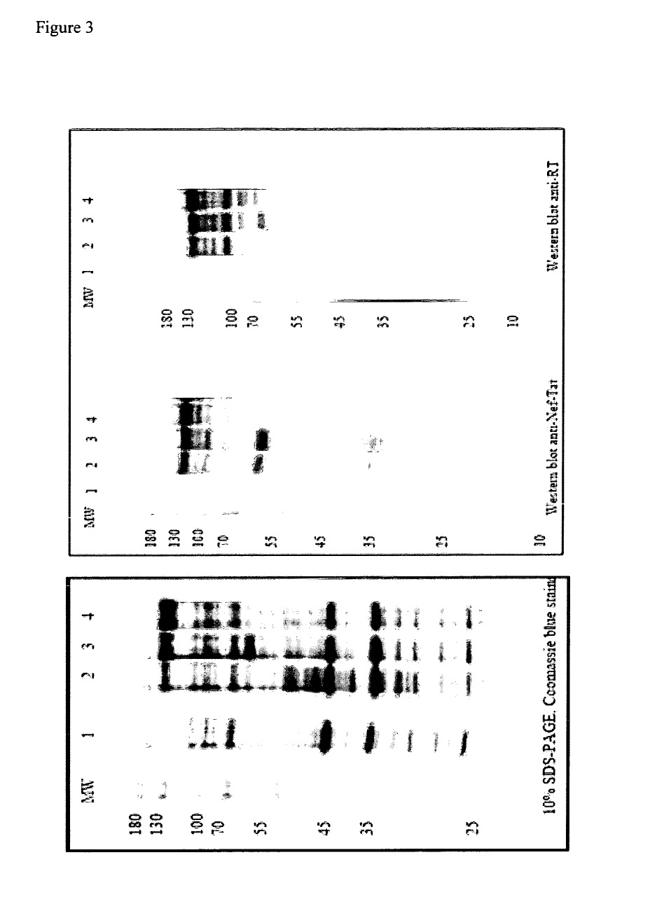

In comparison with F4 from the native gene, the F4 recombinant product profile from the codon-optimised gene is slightly simplified. The major F4-related band at 60 kDa, as well as minor bands below, disappeared (see FIG. 3). Compared to the B834(DE3) recombinant strain expressing F4, the BLR(DE3) strain producing F4co has the following advantages: higher production of F4 full-length protein, less complex band pattern of recombinant product.

FIG. 3 shows coomasie stained gel and western blot for F4 codon-optimized, where

1/ non induced

2/ B834(DE3)/F4 (native gene)

3/ BLR(DE3)/F4 (native gene)

4/ BLR(DE3)/F4 (codon-optimized gene)

### Example 2

### Construction and Expression of P51 RT (Truncated, Codon-Optimised RT)

The RT/p66 region between amino acids 428-448 is susceptible to E. coli proteases. The P51 construct terminates at Leu 427 resulting in the elimination of RNaseH domain.

The putative E. coli “frameshift” sequences identified in RT native gene sequence were also eliminated (by codon-optimization of p51 gene).

p51 Synthetic Gene Design/Construction:

The sequence of the synthetic p51 gene was designed according to E. coli codon usage. Thus it was codon optimized such that the codon usage resembles the codon usage in a highly expressed gene in E. coli. The synthetic gene was constructed as follows: 32 oligonucleotides were assembled in a single-step PCR. In a second PCR the full-length assembly was amplified using the ends primers and the resulting PCR product was cloned into pGEM-T intermediate plasmid. After correction of point errors introduced during gene synthesis, the p51 synthetic gene was cloned into pET29a expression plasmid. This recombinant plasmid was used to transform B834 (DE3) cells.

Recombinant Protein Characteristics:

P51 RT Nucleotide Sequence

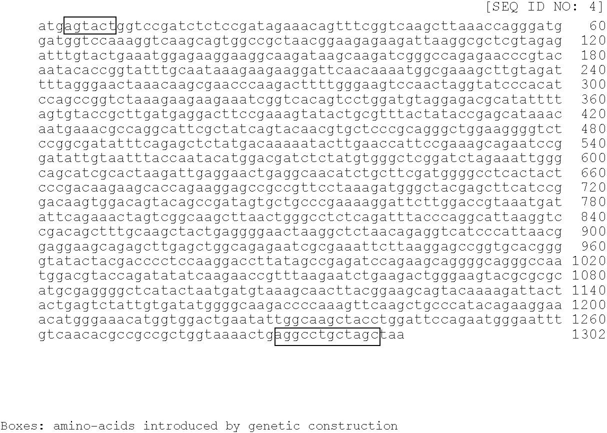

1.1 Amino-Acid Sequence:

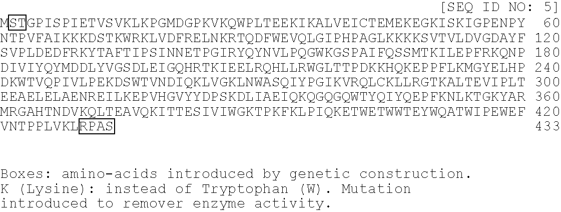

Length, Molecular Weight, Isoelectric Point (IP):
433 AA, MW: 50.3 kDa, IP: 9.08

1.2 p51 Expression in B834(DE3) Cells:

P51 expression level and recombinant protein solubility were evaluated, in parallel to RT/p66 production strain.

p51 Expression Level:

Induction condition: cells grown/induced at 37° C. (+1 mM IPTG), during 5 hours.

Breaking buffer: 50 mM Tris/HCl, pH: 7.5, 1 mM EDTA, +/−1 mM DTT.

Western blot analysis:

Reagents: rabbit polyclonal anti RT (rabbit PO3L16) (dilution: 1/10,000)
Alkaline phosphatase-conjugate anti-rabbit antibody (dilution: 1/7500) Cellular fractions corresponding to crude extracts (T), insoluble pellet (P) and supernatant (S) were run on 10% reducing SDS-PAGE.

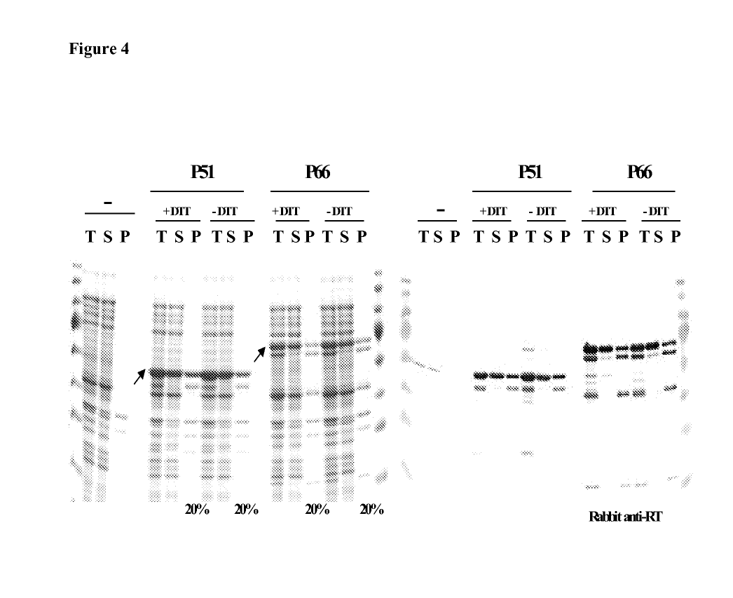

As illustrated on Coomassie stained gel and Western Blot (FIG. 4) very high expression of P51 (15-20% of total protein) was observed, higher than that observed for P66.

For both p51 and p66 proteins (after 5 h induction at 37° C.), 80% of the recombinant products were recovered in the soluble fraction (S1) of cellular extracts (See FIG. 4). When expressed at 30° C., 99% of recombinant proteins were associated with the soluble fraction (data not shown).

The p51 Western Blot pattern was multiband, but less complex than that observed for P66.

Solubility Assay

Solubility assay: Freezing/thawing of Soluble (S1) fraction (5 h induction, 37° C.) prepared under reducing (breaking buffer with DTT) and non-reducing conditions. After thawing, S1 samples were centrifuged at 20.000 g/30 minutes, generating S2 and P2 (p2 is resuspended in 1/10 vol.).

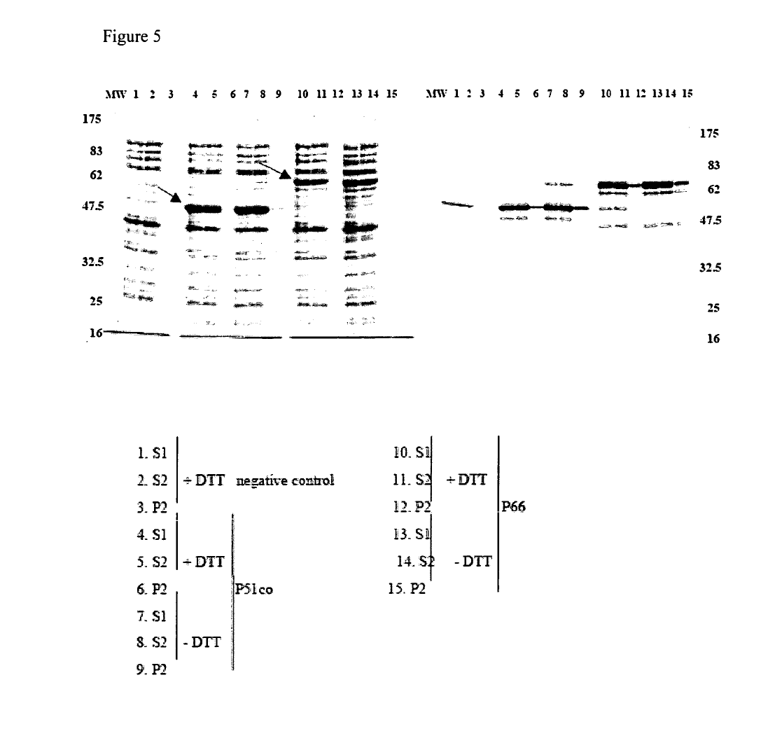

After freezing/thawing of soluble fractions (S1), prepared under reducing as well as non-reducing conditions, 99% of p51 and p66 are still recovered in soluble (S2) fraction. Only 1% is found in the precipitate (P2). This is shown in FIG. 5.

FIG. 5 shows RT/p51 and RT/p66 solubility assay where S1 is soluble fraction (3 h induction at 30° C. conserved at −20° C., After thawing, S1 samples were centrifuged at 20.000 g/30 minutes, generating S2 and P2 (p2 is resuspended in 1/10 vol.).

### Example 3

### Construction and Expression of Nef-p17

The double fusion proteins were constructed
Nef-P17

Recombinant Plasmids Construction:
pET29a/Nef-p17 expression vector:
Nef-p17 fusion gene was amplified by PCR from the F4 recombinant plasmid.

The PCR product was cloned into the intermediate pGEM-T cloning vector and subsequently into the pET29a expression vector.

Recombinant Protein Characteristics:
Length, Molecular Weight, Isoelectric Point (IP)
   Nef-p17 (named NP): 340 AA, MW: 38.5 kDa, IP:7.48
Amino-acid sequences and polynucleotide sequences:

| Nef-p17 nucleotide sequence | |
|---|---|
| [SEQ ID NO: 6] | |
| Atgggtggcaagtggtcaaaaagtagtgtggttggatggcctactgtaagggaaagaatg | 60 |
| Agacgagctgagccagcagcagatggggtgggagcagcatctcgagacctggaaaaacat | 120 |
| Ggagcaatcacaagtagcaatacagcagctaccaatgctgcttgtgcctggctagaagca | 180 |
| Caagaggaggaggaggtgggttttccagtcacacctcaggtacctttaagaccaatgact | 240 |
| Tacaaggcagctgtagatcttagccactttttaaaagaaaaggggggactggaagggcta | 300 |
| Attcactcccaacgaagacaagatatccttgatctgtggatctaccacacacaaggctac | 360 |
| Ttccctgattggcagaactacacaccagggccaggggtcagatatccactgacctttgga | 420 |
| Tggtgctacaagctagtaccagttgagccagataaggtagaagaggccaataaaggagag | 480 |
| Aacaccagcttgttacaccctgtgagcctgcatggaatggatgaccctgagagagaagtg | 540 |
| Ttagagtggaggtttgacagccgcctagcatttcatcacgtggcccgagagctgcatccg | 600 |
| Gagtacttcaagaactgcaggcctatgggtgcgagagcgtcagtattaagcgggggagaa | 660 |
| Ttagatcgatgggaaaaaattcggttaaggccagggggaaagaaaaaatataaattaaaa | 720 |
| Catatagtatgggcaagcagggagctagaacgattcgcagttaatcctggcctgttagaa | 780 |
| Acatcagaaggctgtagacaaatactgggacagctacaaccatcccttcagacaggatca | 840 |
| Gaagaacttagatcattatataatacagtagcaaccctctattgtgtgcatcaaaggata | 900 |
| Gagataaaagacaccaaggaagctttagacaagatagaggaagagcaaaacaaaagtaag | 960 |
| Aaaaaagcacagcaagcagcagctgacacaggacacagcaatcaggtcagccaaaattac | 1020 |
| Taa | 1023 |
| Nef-p17 (NP) | |
| [SEQ ID NO: 7] | |
| MGGKWSKSSVVGWPTVRERMRRAEPAADGVGAASRDLEKHGAITSSNTAATNAACAWLEA | 60 |
| QEEEEVGFPVTPQVPLRPMTYKAAVDLSHFLKEKGGLEGLIHSQRRQDILDLWIYHTQGY | 120 |
| FPDWQNYTPGPGVRYPLTFGWCYKLVPVEPDKVEEANKGENTSLLHPVSLHGMDDPEREV | 180 |
| LEWREDSRLAFHHVARELHPEYFKNC [symbol] IMGARASVLSGGELDRWEKIRLRPGGKKKYKLK | 240 |
| HIVWASRELERFAVNPGLLETSEGCRQILGQLQPSLQTGSEELRSLYNTVATLYCVHQRI | 300 |
| EIKDTKEALDKIEEEQNKSKKKAQQAAADTGHSNQVSQNY | 340 |
| Box: amino-acids introduced by genetic construction. | |
| Nef sequence is in bold. | |
| P17-Nef nucleotide sequence: | |
| [SEQ ID NO: 8] | |
| Atgggtgcgagagcgtcagtattaagcgggggagaattagatcgatgggaaaaaattcgg | 60 |
| Ttaaggccagggggaaagaaaaaatataaattaaaacatatagtatgggcaagcagggag | 120 |
| Ctagaacgattcgcagttaatcctggcctgttagaaacatcagaaggctgtagacaaata | 180 |
| Ctgggacagctacaaccatcccttcagacaggatcagaagaacttagatcattatataat | 240 |
| Acagtagcaaccctctattgtgtgcatcaaaggatagagataaaagacaccaaggaagct | 300 |
| Ttagacaagatagaggaagagcaaaacaaaagtaagaaaaaagcacagcaagcagcagct | 360 |
| Gacacaggacacagcaatcaggtcagccaaaattacctcgacaggcctatgggtggcaag | 420 |
| Tggtcaaaaagtagtgtggttggatggcctactgtaagggaaagaatgagacgagctgag | 480 |
| Ccagcagcagatggggtgggagcagcatctcgagacctggaaaaacatggagcaatcaca | 540 |
| Agtagcaatacagcagctaccaatgctgcttgtgcctggctagaagcacaagaggaggag | 600 |
| Gaggtgggttttccagtcacacctcaggtacctttaagaccaatgacttacaaggcagct | 660 |
| Gtagatcttagccactttttaaaagaaaaggggggactggaagggctaattcactcccaa | 720 |
| Cgaagacaagatatccttgatctgtggatctaccacacacaaggctacttccctgattgg | 780 |
| Cagaactacacaccagggccaggggtcagatatccactgacctttggatggtgctacaag | 840 |
| Ctagtaccagttgagccagataaggtagaagaggccaataaaggagagaacaccagcttg | 900 |
| Ttacaccctgtgagcctgcatggaatggatgaccctgagagagaagtgttagagtggagg | 960 |
| Tttgacagccgcctagcatttcatcacgtggcccgagagctgcatccggagtacttcaag | 1020 |
| Aactgctaa | 1029 |

### Example 4

### Construction and Expression of p24-RT*-Nef-p17 (F4*)

F4* is a mutated version of the F4 (p24-RT/p66-Nef-p17) fusion where the Methionine at position 592 is replaced by a Lysine. This methionine is a putative internal transcriptional “start” site, as supported by N-terminal sequencing performed on a Q sepharose eluate sample of F4 purification experiment. Indeed, the major F4-related small band at 62 kDa present in the Q eluate sample starts at methionine 592.

Methionine is replaced by a lysine: RMR→RKR. The RKR motif is naturally present in clade A RT sequences.

The impact of this mutation on CD4-CD8 epitopes was evaluated:
one HLA-A3 CTL epitope (A* 3002) is lost, but 9 other HLA-A3 epitopes are present in the RT sequence.
No helper epitope identified in this region.

Recombinant Protein Characteristics:

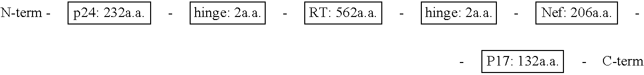

Length, Molecular Weight, Isoelectric Point (IP):
   1136 AA, 129 kDa, IP: 8.07
Nucleotide sequence:

[SEQ ID NO: 9]
atggttatcgtgcagaacatccaggggcaaatggtacatcaggccatatc
acctagaactttaaatgcatgggtaaaagtagtagaagagaaggctttca
gcccagaagtaatacccatgttttcagcattatcagaaggagccacccca
caagatttaaacaccatgctaaacacagtggggggacatcaagcagccat
gcaaatgttaaaagagaccatcaatgaggaagctgcagaatgggatagag
tacatccagtgcatgcagggcctattgcaccaggccagatgagagaacca
aggggaagtgacatagcaggaactactagtacccttcaggaacaaatagg
atggatgacaaataatccacctatcccagtaggagaaatttataaaagat
ggataatcctgggattaaataaaatagtaagaatgtatagccctaccagc
attctggacataagacaaggaccaaaagaaccttttagagactatgtaga
ccggttctataaaactctaagagccgagcaagcttcacaggaggtaaaaa
attggatgacagaaaccttgttggtccaaaatgcgaacccagattgtaag
actattttaaaagcattgggaccagcggctacactagaagaaatgatgac
agcatgtcagggagtaggaggacccggccataaggcaagagttttgtcat
atgggccccattagccctattgagactgtgtcagtaaaattaaagccagg
aatggatggcccaaaagttaaacaatggccattgacagaagaaaaaataa
aagcattagtagaaatttgtacagagatggaaaaggaagggaaaatttca
aaaattgggcctgaaaatccatacaatactccagtatttgccataaagaa
aaaagacagtactaaatggagaaaattagtagatttcagagaacttaata
agagaactcaagacttctgggaagttcaattaggaataccacatcccgca
gggttaaaaaagaaaaaatcagtaacagtactggatgtgggtgatgcata
tttttcagttcccttagatgaagacttcaggaaatatactgcatttacca
tacctagtataaacaatgagacaccagggattagatatcagtacaatgtg
cttccacagggatggaaaggatcaccagcaatattccaaagtagcatgac
aaaaatcttagagccttttagaaaacaaaatccagacatagttatctatc
aatacatggatgatttgtatgtaggatctgacttagaaatagggcagcat
agaacaaaaatagaggagctgagacaacatctgttgaggtggggacttac
cacaccagacaaaaaacatcagaaagaacctccattccttaaaatgggtt
atgaactccatcctgataaatggacagtacagcctatagtgctgccagaa
aaagacagctggactgtcaatgacatacagaagttagtggggaaattgaa
ttgggcaagtcagatttacccagggattaaagtaaggcaattatgtaaac
tccttagaggaaccaaagcactaacagaagtaataccactaacagaagaa
gcagagctagaactggcagaaaacagagagattctaaaagaaccagtaca
tggagtgtattatgacccatcaaaagacttaatagcagaaatacagaagc
aggggcaaggccaatggacatatcaaatttatcaagagccatttaaaaat
ctgaaaacaggaaaatatgcacgtaaacgcggtgcccacactaatgatgt
aaaacaattaacagaggcagtgcaaaaaataaccacagaaagcatagtaa
tatggggaaagactcctaaatttaaactgcccatacaaaaggaaacatgg
gaaacatggtggacagagtattggcaagccacctggattcctgagtggga
gtttgttaatacccctcctttagtgaaattatggtaccagttagagaaag
aacccatagtaggagcagaaaccttctatgtagatggggcagctaacagg
gagactaaattaggaaaagcaggatatgttactaatagaggaagacaaaa
agttgtcaccctaactgacacaacaaatcagaagactgagttacaagcaa
tttatctagctttgcaggattcgggattagaagtaaacatagtaacagac
tcacaatatgcattaggaatcattcaagcacaaccagatcaaagtgaatc
agagttagtcaatcaaataatagagcagttaataaaaaaggaaaaggtct
atctggcatgggtaccagcacacaaaggaattggaggaaatgaacaagta
gataaattagtcagtgctggaatcaggaaagtgcta [symbol] ggtggcaa
gtggtcaaaaagtagtgtggttggatggcctactgtaagggaaagaatga
gacgagctgagccagcagcagatggggtgggagcagcatctcgagacctg
gaaaaacatggagcaatcacaagtagcaatacagcagctaccaatgctgc
ttgtgcctggctagaagcacaagaggagg
aggaggtgggttttccagtcacacctcaggtacctttaagaccaatgact
tacaaggcagctgtagatcttagccactttttaaaagaaaaggggggact
ggaagggctaattcactcccaacgaagacaagatatccttgatctgtgga
tctaccacacacaaggctacttccctgattggcagaactacacaccaggg
ccaggggtcagatatccactgacctttggatggtgctacaagctagtacc
agttgagccagataaggtagaagaggccaataaaggagagaacaccagct
tgttacaccctgtgagcctgcatggaatggatgaccctgagagagaagtg
ttagagtggaggtttgacagccgcctagcatttcatcacgtggcccgaga
gctgcatccggagtacttcaagaactgc [symbol] atgggtgcgagagcgt
cagtattaagcgggggaga
attagatcgatgggaaaaaattcggttaaggccagggggaaagaaaaaat
ataaattaaaacatatagtatgggcaagcagggagctagaacgattcgca
gttaatcctggcctgttagaaacatcagaaggctgtagacaaatactggg
acagctacaaccatcccttcagacaggatcagaagaacttagatcattat
ataatacagtagcaaccctctattgtgtgcatcaaaggatagagataaaa
gacaccaaggaagctttagacaagatagaggaagagcaaaacaaaagtaa
gaaaaaagcacagcaagcagcagctgacacaggacacagcaatcaggtca
gccaaaattactaa
p24 sequence is in bold
Nef sequence is underlined
Boxes: nucleotides introduced by genetic
construction

Amino-Acid Sequence

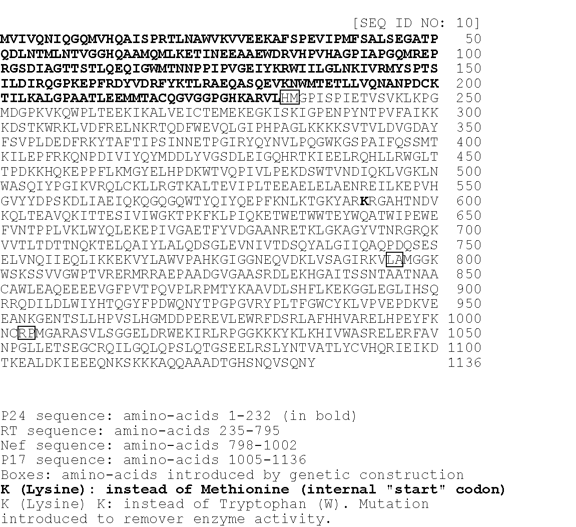

F4* expression in B834(DE3) cells:

F4* recombinant strain was induced at 22° C. during 18 h, in parallel to F4 non-mutated construct. Crude extracts were prepared and analyzed by Coomassie stained gel and Western blotting.

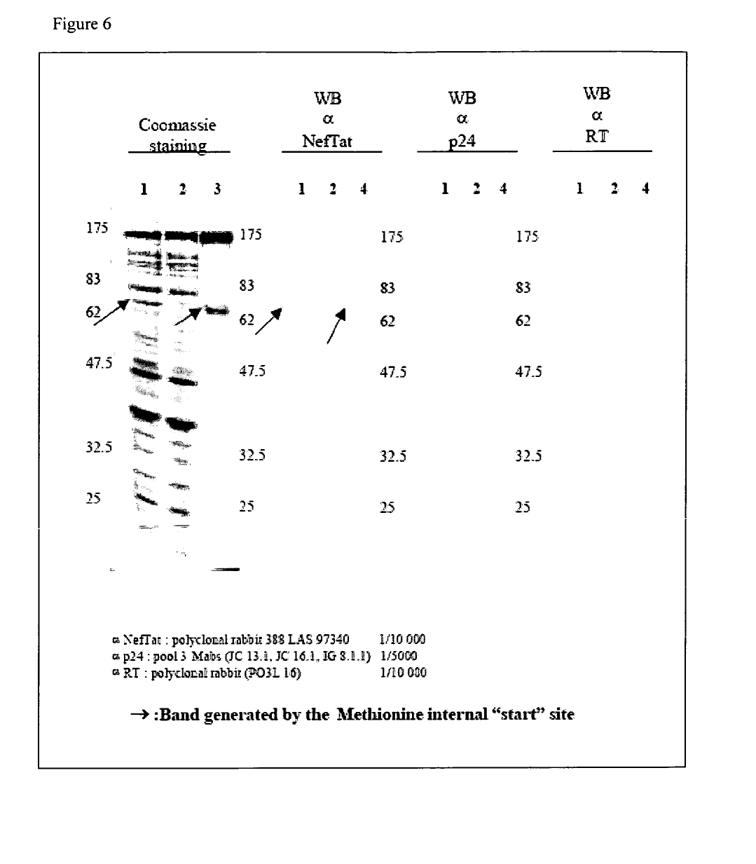

As illustrated in FIG. 6, F4* was expressed at a high level (10% total protein), slightly higher compared to F4 and the small 62 kDa band disappeared.

FIG. 6 shows SDS-PAGE analysis under reducing condition (10% SDS-PAGE reducing gel; Induction: 19 hours, 22° C.) for various F4 proteins, where 1 is F4, 2 is F4*, 3 is F4 (Q sepharose elute sample) 2,5 μg and 4 is F4 (Q sepharose elute sample) 250 ng.

Western blot analysis:

Reagents: pool 3 Mabs anti p24 (JC13.1, JC16.1, IG8.1.1)(dilution 1/5000)
rabbit polyclonal anti RT (rabbit PO3L16) (dilution: 1/10 000)
rabbit polyclonal anti Nef-Tat (rabbit 388) (dilution 1/10 000)
   Alkaline phosphatase-conjugate anti-rabbit antibody (dilution: 1/7500)
   Alkaline phosphatase-conjugate anti-mouse antibody (dilution: 1/7500)

Induction condition: cells grown at 37° C./induced at 30° C. (+1 mM IPTG), during 3 h.

Breaking buffers: F4:50 mM Tris/HCl pH: 8.0, 50 mM NaCl, 1 mM EDTA, +/−1 mM DTT

Western blot analysis:

reagents rabbit polyclonal anti RT (rabbit PO3L16) (dilution: 1/10 000)
rabbit polyclonal anti Nef-Tat (rabbit 388) (dilution 1/10 000)
Alkaline phosphatase-conjugate anti-rabbit antibody (dilution: 1/7500)

### Example 5

### Construction and expression of F4(p51) and F4(p51)*

RT/p51 was used in the F4 fusion construct (in place of RT/p66).

F4(p51)=p24-p51-Nef-p17

F4(p51)*=p24-p51*-Nef-p17—Mutated F4(p51): putative internal Methionine initiation site (present in RT portion) replaced by Lysine, to further simplify the antigen pattern.

Recombinant Plasmids Construction:

F4(p51): The sequence encoding p51 was amplified by PCR from pET29a/p51 expression plasmid. Restriction sites were incorporated into the PCR primers (NdeI and StuI at the 5′ end. AvrII at the 3′ end of the coding sequence). The PCR product was cloned into pGem-T intermediate plasmid and sequenced. pGem-T/p51 intermediate plasmid was restricted by NdeI and AvrII and the p51 fragment was ligated into pET28b/p24-RT/p66-Nef-p 17 expression plasmid restricted by NdeI and NheI (resulting in the excision of RT/p66 sequence). Ligation was performed by combining digestion reactions in appropriate concentrations, in the presence of T4 DNA ligase. Ligation product was used to transform DH5α E. coli cells. Verification of insertion of p51 into the correct translational reading frame (in place of RT/p66 in the f4 fusion) was confirmed by DNA sequencing. The resulting fusion construct p24-RT/p51-Nef-p17 is named F4(p51).

F4(p51)*: Mutation of the putative internal methionine initiation site (present in RT/p51) was achieved with “GeneTailor Site-Directed Mutagenesis system” (Invitrogen), generating F4(p51)* construct.

F4(p51) and F4(p51)* expression plasmids were used to transform B834(DE3) cells.

Recombinant Proteins Characteristics:

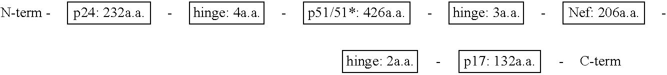

Length, Molecular Weight, Isoelectric Point (IP):
   1005 AA, 114.5 kDa, IP: 8.47
Nucleotide sequence (for F4(p51)*)

| [SEQ ID NO: 11] | |
|---|---|
| Atggttatcgtgcagaacatccaggggcaaatggtacatcaggccatatcacctagaact | 60 |
| Ttaaatgcatgggtaaaagtagtagaagagaaggctttcagcccagaagtaatacccatg | 120 |
| Ttttcagcattatcagaaggagccaccccacaagatttaaacaccatgctaaacacagtg | 180 |
| Gggggacatcaagcagccatgcaaatgttaaaagagaccatcaatgaggaagctgcagaa | 240 |
| Tgggatagagtacatccagtgcatgcagggcctattgcaccaggccagatgagagaacca | 300 |
| Aggggaagtgacatagcaggaactactagtacccttcaggaacaaataggatggatgaca | 360 |
| Aataatccacctatcccagtaggagaaatttataaaagatggataatcctgggattaaat | 420 |
| Aaaatagtaagaatgtatagccctaccagcattctggacataagacaaggaccaaaagaa | 480 |
| Ccttttagagactatgtagaccggttctataaaactctaagagccgagcaagcttcacag | 540 |
| Gaggtaaaaaattggatgacagaaaccttgttggtccaaaatgcgaacccagattgtaag | 600 |
| Actattttaaaagcattgggaccagcggctacactagaagaaatgatgacagcatgtcag | 660 |
| Ggagtaggaggacccggccataaggcaagagttttg [symbol] GGTCCGATCTCT | 720 |
| CCGATAGAAACAGTTTCGGTCAAGCTTAAACCAGGGATGGATGGTCCAAAGGTCAAGCAG | 780 |
| TGGCCGCTAACGGAAGAGAAGATTAAGGCGCTCGTAGAGATTTGTACTGAAATGGAGAAG | 840 |
| GAAGGCAAGATAAGCAAGATCGGGCCAGAGAACCCGTACAATACACCGGTATTTGCAATA | 900 |
| AAGAAGAAGGATTCAACAAAATGGCGAAAGCTTGTAGATTTTAGGGAACTAAACAAGCGA | 960 |
| ACCCAAGACTTTTGGGAAGTCCAACTAGGTATCCCACATCCAGCCGGTCTAAAGAAGAAG | 1020 |
| AAATCGGTCACAGTCCTGGATGTAGGAGACGCATATTTTAGTGTACCGCTTGATGAGGAC | 1080 |
| TTCCGAAAGTATACTGCGTTTACTATACCGAGCATAAACAATGAAACGCCAGGCATTCGC | 1140 |
| TATCAGTACAACGTGCTCCCGCAGGGCTGGAAGGGGTCTCCGGCGATATTTCAGAGCTCT | 1200 |
| ATGACAAAAATACTTGAACCATTCCGAAAGCAGAATCCGGATATTGTAATTTACCAATAC | 1260 |
| ATGGACGATCTCTATGTGGGCTCGGATCTAGAAATTGGGCAGCATCGCACTAAGATTGAG | 1320 |
| GAACTGAGGCAACATCTGCTTCGATGGGGCCTCACTACTCCCGACAAGAAGCACCAGAAG | 1380 |
| GAGCCGCCGTTCCTAAAGATGGGCTACGAGCTTCATCCGGACAAGTGGACAGTACAGCCG | 1440 |
| ATAGTGCTGCCCGAAAAGGATTCTTGGACCGTAAATGATATTCAGAAACTAGTCGGCAAG | 1500 |
| CTTAACTGGGCCTCTCAGATTTACCCAGGCATTAAGGTCCGACAGCTTTGCAAGCTACTG | 1560 |
| AGGGGAACTAAGGCTCTAACAGAGGTCATCCCATTAACGGAGGAAGCAGAGCTTGAGCTG | 1620 |
| GCAGAGAATCGCGAAATTCTTAAGGAGCCGGTGCACAGGGTATACTACGACCCCTCCAAG | 1680 |
| GACCTTATAGCCGAGATCCAGAAGCAGGGGCAGGGCCAATGGACGTACCAGATATATCAA | 1740 |
| GAACCGTTTAAGAATCTGAAGACTGGGAAGTACGCGCGCAAACGAGGGGCTCATACTAAT | 1800 |
| GATGTAAAGCAACTTACGGAAGCAGTACAAAAGATTACTACTGAGTCTATTGTGATATGG | 1860 |
| GGCAAGACCCCAAAGTTCAAGCTGCCCATACAGAAGGAAACATGGGAAACATGGTGGACT | 1920 |
| GAATATTGGCAAGCTACCTGGATTCCAGAATGGGAATTTGTCAACACGCCGCCGCTGGTA | 1980 |
| AAACTG [symbol] ATGggtggcaagtggtcaaaaagtagtgtggttggatggcctact | 2040 |
| Gtaagggaaagaatgagacgagctgagccagcagcagatggggtgggagcagcatctcga | 2100 |
| Gacctggaaaaacatggagcaatcacaagtagcaatacagcagctaccaatgctgcttgt | 2160 |
| Gcctggctagaagcacaagaggaggaggaggtgggttttccagtcacacctcaggtacct | 2220 |
| Ttaagaccaatgacttacaaggcagctgtagatcttagccactttttaaaagaaaagggg | 2280 |
| Ggactggaagggctaattcactcccaacgaagacaagatatccttgatctgtggatctac | 2340 |
| Cacacacaaggctacttccctgattggcagaactacacaccagggccaggggtcagatat | 2400 |
| Ccactgacctttggatggtgctacaagctagtaccagttgagccagataaggtagaagag | 2460 |
| Gccaataaaggagagaacaccagcttgttacaccctgtgagcctgcatggaatggatgac | 2520 |
| Cctgagagagaagtgttagagtggaggtttgacagccgcctagcatttcatcacgtggcc | 2580 |
| Cgagagctgcatccggagtacttcaagaactgc [symbol] ATGGGTGCGAGAGCGTCAGTA | 2640 |
| TTAAGCGGGGGAGAATTAGATCGATGGGAAAAAATTCGGTTAAGGCCAGGGGGAAAGAAA | 2700 |
| AAATATAAATTAAAACATATAGTATGGGCAAGCAGGGAGCTAGAACGATTCGCAGTTAAT | 2760 |
| CCTGGCCTGTTAGAAACATCAGAAGGCTGTAGACAAATACTGGGACAGCTACAACCATCC | 2820 |
| CTTCAGACAGGATCAGAAGAACTTAGATCATTATATAATACAGTAGCAACCCTCTATTGT | 2880 |
| GTGCATCAAAGGATAGAGATAAAAGACACCAAGGAAGCTTTAGACAAGATAGAGGAAGAG | 2940 |
| CAAAACAAAAGTAAGAAAAAAGCACAGCAAGCAGCAGCTGACACAGGACACAGCAATCAG | 3000 |
| GTCAGCCAAAATTACtaa | 3018 |

P24: sequence in bold
P51: sequence in capital letter
Nef: sequence in small letter
P17: sequence underlined
Boxes: nucleotides introduced by genetic construction

Amino-Acid sequence (for F4(p51)*)

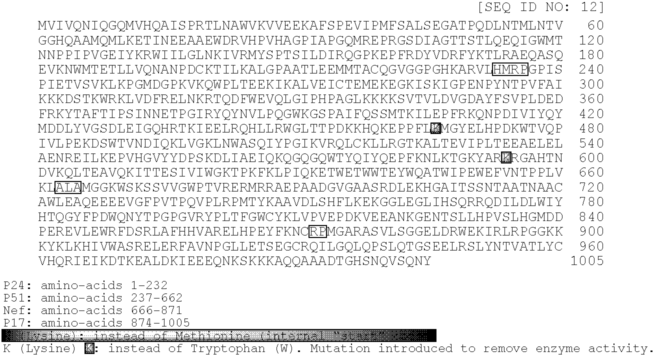

F4(p51) Expression in B834(DE3) Cells:

F4(p51) expression level and recombinant protein solubility were evaluated, in parallel to F4 expressing strain.

Induction condition: cells grown at 37° C./induced at 22° C. (+1 mM IPTG), over 19 h.

Breaking buffer: 50 mM Tris/HCl pH: 7.5, 1 mM EDTA, 1 mM DTT

Western blot analysis:

reagents rabbit polyclonal anti RT (rabbit PO3L16) (dilution: 1/10 000)
rabbit polyclonal anti Nef-Tat (rabbit 388) (dilution 1/10 000)
Alkaline phosphatase-conjugate anti-rabbit antibody (dilution: 1/7500)

Cellular fractions corresponding to crude extracts (T), insoluble pellet (P) and supernatant (S) were analyzed on 10% reducing SDS-PAGE.

F4(p51) was expressed at a high level (10% of total protein), similar to F4. Almost all F4(p51) is recovered in the soluble fraction (S) of cellular extracts. Upon detection with an anti-Nef-tat reagent, F4(p51) the WB pattern was shown to be simplified (reduction of truncated products below +/−60 kDa).

F4(p51)* Expression in B834(DE3) Cells:

F4(p51)* recombinant strain was induced at 22° C. over 18 h, in parallel to F4(p51) non-mutated construct, F4 and F4*. Crude cellular extracts were prepared and analyzed by Coomassie stained gel and Western blotting. High expression of F4(p51) and F4(p51)* fusions was observed, representing at least 10% of total protein. WB pattern: reduction of truncated products below +/−60 kDa. In addition, for F4(p51)* construct, the 47 kDa band (due to internal start site) has disappeared.

### Example 6

### Purification of F4, F4(p51)* and F4*—Purification Method I

The fusion protein F4, comprising the 4 HIV antigens p24-RT-Nef-p17, was purified from a E. coli cell homogenate according to purification method I, which comprises the following principal steps:
Ammonium sulfate precipitation of F4
SO₃ Fractogel cation-exchange chromatography (positive mode)
Octyl sepharose hydrophobic interaction chromatography (positive mode)
Q sepharose FF anion-exchange chromatography (positive mode)
Superdex 200 gel filtration chromatography in presence of SDS
Dialysis and concentration

Additionally, the F4(p51)* fusion protein (RT replaced by the codon optimized p51 carrying an additional mutation Met592Lys) and the F4* protein (F4 carrying an additional Met592Lys mutation) were purified using the same purification method I.

Protein Quantification
Total protein was determined using the Lowry assay. Before measuring the protein concentration all samples are dialyzed overnight against PBS, 0.1% SDS to remove interfering substances (urea, DTT). BSA (Pierce) was used as the standard.

SDS-PAGE and Western Blot
Samples were prepared in reducing or non-reducing SDS-PAGE sample buffer (+/−β-mercaptoethanol) and heated for 5 min at 95° C.
Proteins were separated on 4-20% SDS-polyacrylamide gels at 200 V for 75 min using pre-cast Novex Tris-glycine gels or Criterion gels (Bio-Rad), 1 mm thick.
Proteins were visualized with Coomassie-blue R250.
For the western blots (WB), the proteins were transferred from the SDS-gel onto nitrocellulose membranes (Bio-Rad) at 4° C. for 1.5 h at 100 V or overnight at 30 V.
F4 was detected using monoclonal antibodies against the different antigens, anti-p24, anti-Nef-Tat, anti-RT (sometimes a mixture of anti-p24 and anti Nef-Tat was used to detect a maximum number of protein bands).
Alkaline-phosphatase conjugated anti-mouse or anti-rabbit antibodies were bound to the primary antibodies and protein bands were visualized using BCIP and NBT as the substrates.

anti-E. coli Western Blot
5 μg protein (Lowry) were separated by SDS-PAGE and transferred onto nitrocellulose membranes as above.
Residual host cell proteins were detected using polyclonal anti-E. coli antibodies. Protein bands were visualized with the alkaline-phosphatase reaction as above.

Purification Method I

Method I comprises a precipitation by ammonium sulfate and four chromatographic steps:
E. coli cells were homogenized in 50 mM Tris buffer at pH 8.0 in the presence of 10 mM DTT, 1 mM PMSF, 1 mM EDTA at OD50 (˜360 ml). 2 Rannie passages were applied at 1000 bars.
Cells debris and insoluble material were removed by centrifugation at 14400×g for 20 min.
Ammonium sulfate (AS) was added from a 3.8M stock solution to the clarified supernatant to a final concentration of 1.2M. Proteins were precipitated for ˜2 hours at room temperature (RT) and then pelleted by centrifugation (10 min at 14400×g). The pellet was resuspended in 8M urea, 10 mM DTT in 10 mM phosphate buffer at pH 7.0.
The antigen was captured on a S03 Fractogel column (Merck) in the presence of 8M urea and 10 mM DTT at pH 7.0 in phosphate buffer. The column was washed to elute non-bound protein followed by a pre-elution step with 170 mM NaCl to remove bound host cell proteins (HCP). F4 was then eluted with 460 mM NaCl, 8M urea, 10 mM DTT in phosphate buffer at pH 7.0.
The SO₃ eluate was 2 fold diluted with 10 mM phosphate buffer, pH 7, and loaded onto a Octyl sepharose column (Amersham Biosciences) in the presence of 4M urea, 1 mM DTT, 230 mM NaCl in phosphate buffer at pH 7.0. Following a washing step (equilibration buffer) bound F4 was eluted with 8M urea, 1 mM DTT in 25 mM Tris buffer at pH 8.0.
The Octyl eluate was diluted and adjusted to pH 9.0 and F4 was then bound to an Q sepharose column (Amersham Bioscience) in the presence of 8M urea at pH 9.0 (25 mM Tris). Unbound protein was washed off (8M urea, 25 mM Tris at pH 9.0) and a pre-elution step (90 mM NaCl in 8M urea, 25 mM Tris, pH 9.0) removed HCP and F4-degradation products. F4 was desorped from the column with 200 mM NaCl, 8M urea in Tris buffer at pH 9.0.
An aliquot of the Q eluate was spiked with 1% SDS and dialyzed against PBS buffer containing 0.1% SDS and 1 mM DTT to remove the urea prior to injecting the sample onto the gel filtration column (prep grade Superdex 200, two 16×60 cm columns connected in a row). The relevant fractions were pooled after in-process SDS-PAGE analysis.
Samples were dialyzed twice at RT in dialysis membranes (12-14 kDa cut-off) overnight against 1 1 0.5M Arginine, 10 mM Tris, 5 mM Glutathione, pH 8.5.

The sequential purification steps are shown in the flowchart below.

### Example 7

### Purification of F4 and F4co (Codon Optimized)—Purification Method II

Purification Method II

A simplified purification procedure, method II as compared to method I, was also developed. Method II consists of only 2 chromatographic steps and a final dialysis/diafiltration for buffer exchange. Notably, a CM hyperZ chromatographic column (BioSepra) was introduced to replace the clarification step, the ammonium sulfate precipitation and the SO₃ chromatography of method I (See Example 6). Method II was used to purify both F4 and full-codon optimized F4 (“F4co”). For F4co, two different forms of method II were performed, one involving carboxyamidation and one not. The purpose of the carboxyamidation step was to prevent oxidative aggregation of the protein. This carboxyamidation is performed after the 1^{st} chromatographic step (CM hyperZ).
E. coli cells (expressing F4 or F4co) were homogenized in 50 mM Tris buffer at pH 8.0 in the presence of 10 mM DTT, at OD90. 2 Rannie passages were applied at 1000 bars.
8M urea were added to the homogenate before application to the CM hyperZ resin (BioSepra) equilibrated with 8M urea in phosphate buffer at pH 7. Antigen capture was done in a batch mode. The resin was then packed in a column, unbound proteins were washed off with the equilibration buffer and bound host cell proteins (HCP) were removed by a pre-elution step with 120 mM NaCl. F4co was then eluted with 360 mM NaCl, 8M urea, 10 mM DTT in phosphate buffer at pH 7.0.
To control oxidative aggregation of the fusion protein, the cysteine groups of F4co can be carboxyamidated with idoacetamide. Therefore, optionally, 50 mM iodoacetamide was added to the CM hyperZ eluate and carboxyamidation was done for 30 min at room temperature in the dark.
The CM hyperZ eluate was then adequately diluted (about 5-8 fold) and adjusted to pH 9.0. F4co or F4coca (codon optimized carboxyamidated) was then bound to a Q sepharose column (Amersham Bioscience) in the presence of 8M urea in Tris buffer at pH 9.0. Unbound protein was washed off with the equilibration buffer and a pre-elution step with 90 mM NaCl (only with non-carboxyamidated protein) in the same buffer removed bound HCP. F4co was desorped from the column with 200 mM NaCl, 8M urea in Tris buffer at pH 9.0.
Samples were dialyzed twice at RT in dialysis membranes (12-14 kDa cut-off) overnight against 1 1 0.5M Arginine, 10 mM Tris buffer, 10 mM Glutathione (only added to the non-carboxyamidated protein), pH 8.5. Alternatively, buffer exchange was accomplished by diafiltration against 10 sample volumes of the same buffer using a tangential-flow membrane with 30 or 50 kDa cut-off.
Finally, the dialyzed product was sterile filtered through a 0.22 μm membrane.

The sequential purification steps are shown in the flowchart below.

Results: Purification of F4co

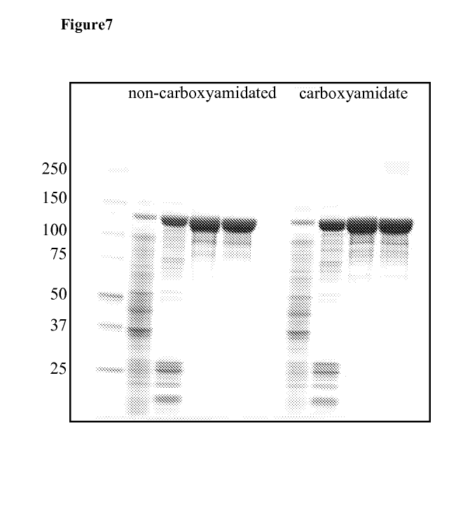

FIG. 7 shows a SDS gel of the F4-containing fractions collected during the purification of F4co and the purification of carboxyamidated F4co (“F4coca”).

The CM hyperZ resin completely captured F4co from the crude homogenate (lane 1) in the presence of 8M urea and quantitative elution was achieved with 360 mM NaCl. The CM hyperZ eluate shown in lane 2 was considerably enriched in F4co. After appropriate dilution and adjustment of the sample to pH 9, F4co or F4coca was bound to a Q sepharose column. F4co or F4coca was then specifically eluted with 200 mM NaCl as shown in lane 3. This chromatography not only removed remaining host cell proteins but also DNA and endotoxins. To bring the purified material in a formulation-compatible buffer, the Q sepharose eluate was dialyzed against 10 mM Tris buffer, 0.5M Arginine, 10 mM Glutathione pH 8.5 in a dialysis membrane with 12-14 kDa cut-off. Glutathione was omitted with the carboxyamidated protein.

Purification of both F4co and F4coca yielded about 500 mg purified material per L of culture OD130. This was in a similar range as observed before with the non-codon-optimized F4.

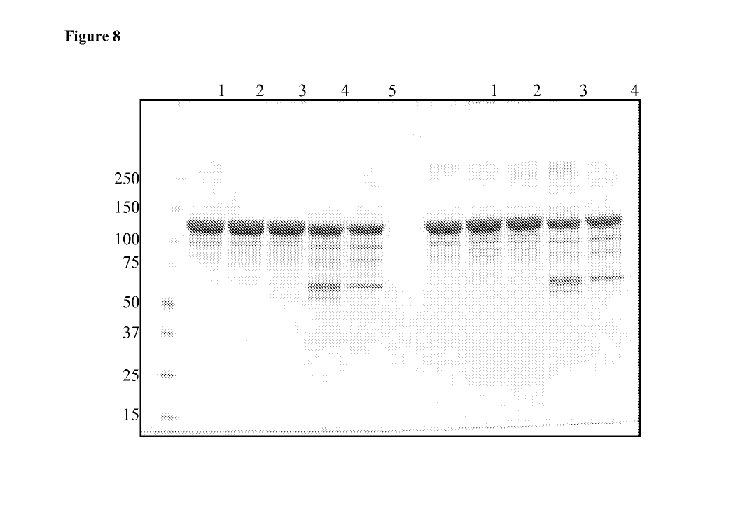

As described above, two different purification methods (I and II) have been developed to purify the different F4 constructs. FIG. 8 compares the different purified bulks that were obtained.

F4 presented several strong low molecular weight (LMW) bands, only faint bands were visible with the codon-optimized F4co. Method I and method II produce a very similar F4co pattern. Anti-E. coli western blot analysis confirmed the purity of the purified proteins indicating host cell protein contamination below 1% in all the preparations.

### Example 8

Two antioxidant mechanisms that could avoid oxidation were tested:

Chelating agents:

Chelating agents may in some formulation be able to chelate ions present in the formulation, which may catalyze of the oxidation reactions. This was tested for formulations containing proteins employed in the present invention.

—SH containing compounds:

The —SH functions of those antioxidants may stabilize the protein after reaction with the —SH functions of F4co or may be oxidized instead of —SH functions of the protein. Four chelating agents were tested namely: citric acid trisodium salt, malic acid sodium salt, dextrose, L-methionine and four antioxidants were tested namely glutathione, cysteine, N-acetyl cysteine, and monothioglycerol.

The efficacy of the selected agents was evaluated according to their capacity to avoid intermolecular and/or intramolecular oxidation of F4co. Results obtained for tested antioxidants were compared to those obtained with sodium sulfite (reducing agent)+EDTA (chelating agent) where only intramolecular oxidation is avoided.

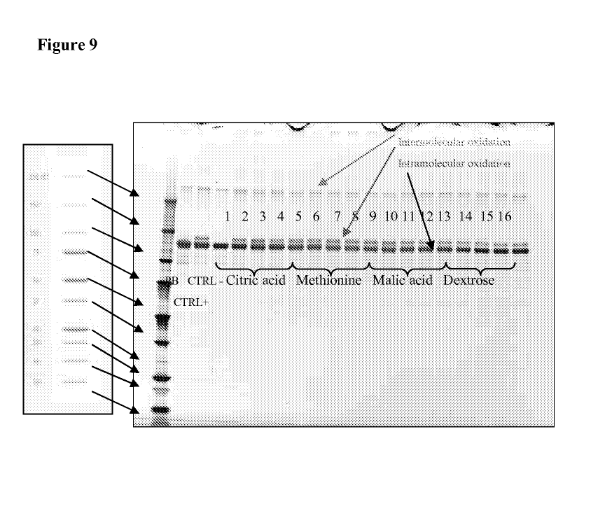

FIG. 9 shows the screening of chelating agents citric acid, L-methionine, malic acid and dextrose analysis by SDS PAGE in non-reducing conditions under non-reducing conditions, where:

| | | | |
|---|---|---|---|
| 1 | Citric acid trisodium salt | 0.5% | w/v |
| 2 | Citric acid trisodium salt | 1.0% | w/v |
| 3 | Citric acid trisodium salt | 1.5% | w/v |
| 4 | Citric acid trisodium salt | 2.0% | w/v |
| 5 | L-methionine | 0.001% | w/v |
| 6 | L-methionine | 0.01% | w/v |
| 7 | L-methionine | 0.1% | w/v |
| 8 | L-methionine | 0.5% | w/v |
| 9 | Malic acid sodium salt | 0.001% | w/v |
| 10 | Malic acid sodium salt | 0.01% | w/v |
| 11 | Malic acid sodium salt | 0.1% | w/v |
| 12 | Malic acid sodium salt | 0.5% | w/v |
| 13 | Dextrose | 0.001% | w/v |
| 14 | Dextrose | 0.01% | w/v |
| 15 | Dextrose | 0.1% | w/v |
| 16 | Dextrose | 1.0% | w/v |

The screening of antioxidants was executed in 2 steps. First, the 8 agents were submitted to a pre-screening on the Final Bulk 30 μg dose. Then, according to the results, the efficient antioxidants underwent screening on the Final Bulk and Final Container 90 μg dose.
a. Pre-screening on 30 μg dose (Final Bulk)

The pre-screening testing on the 30 μg dose was analyzed on a SDS-PAGE in non-reducing conditions on the Final Bulk stored 1 day at 4° C.
b. Screening on 90 μg dose

Potential antioxidants screened were further analyzed in the 90 μg formulation to analyze the efficacy through the different formulation steps including storage of Final Bulk, filling, freeze-drying and reconstitution.

Antigen Solubility
Visual observation

Formulations (500 μl) were observed in cuvette in front of the natural light. Formulations were described as ‘clear’ (transparent solution) or ‘turbid’.
Centrifugation (14300 g 15 min) followed by SDS-PAGE in reducing conditions

No negative impact observed on F4co solubility for cysteine, N-acetylcysteine or monothioglycerol or glutathione.

Antigen Oxidation

SDS-PAGE in Non Reducing Conditions

Formulated protein was compared to the purified bulk, to a negative control (F4co formulated with EDTA and sodium sulfite) and to a positive control (F4co formulated without addition of sodium sulfite and EDTA).

Stability and Accelerated Stability Testing

Final Bulk:
SDS-PAGE in NON REDUCING conditions at T1 (day 1), T8 (day 8) and, T15 (day 15) after storage at 4° C.

Final Container reconstituted:
SDS PAGE in NON REDUCING conditions after reconstitution of cakes in water after freeze-drying (TO) or after storage 7 days 37° C.* or under AOT**.
SDS-PAGE in NON REDUCING conditions 24hours after reconstitution in a liposomal adjuvant at 25° C.
SDS-PAGE in REDUCING conditions on the Final container reconstituted in liposomal adjuvant after 4 hours stored at 25° C.
   *7 days 37° C.
   Freeze-dried cakes have been submitted to a temperature of 37° C. during 7 days in order to accelerate stability. After, cakes were reconstituted in water for injection in order to be analyzed by SDS-PAGE in NON REDUCING conditions.

### **Accelerated Oxidation Test (AOT)

Freeze-dried cakes have been submitted to a light of 765 w/m² for 15 hours in order to force exposition of product to light. After, cakes were reconstituted in water for injection in order to be analyzed by SDS-PAGE in NON REDUCING conditions.
a) Formulation flow-sheet

The various formulations were prepared in accordance with the flow-sheet below, but sodium sulfite has been replaced by the relevant antioxidants.

Results

Screening of —SH Containing Compounds

Formulations containing —SH functions: glutathione, monothioglycerol, cysteine and N-acetylcysteine were analyzed.
Pre-screening on 30 μg dose (Final Bulk)

The —SH containing compounds exhibited promising results at Final Bulk step after l day at 4° C.: neither intramolecular or intermolecular oxidation was observed at the highest concentration tested (0.625%).
Screening on 90 μg dose
   Final Bulk Stability

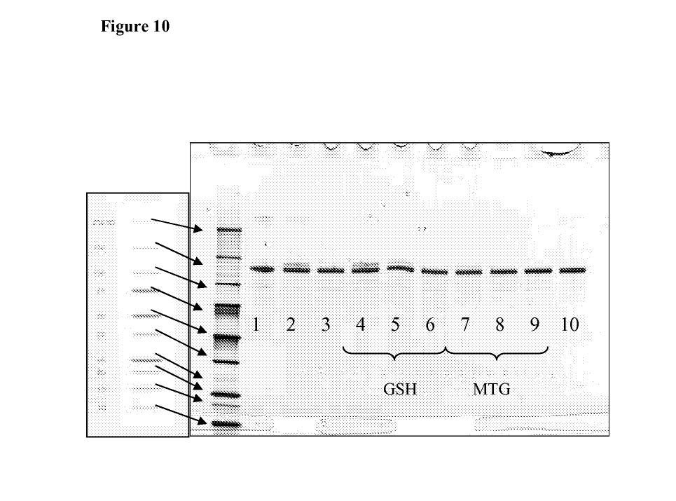

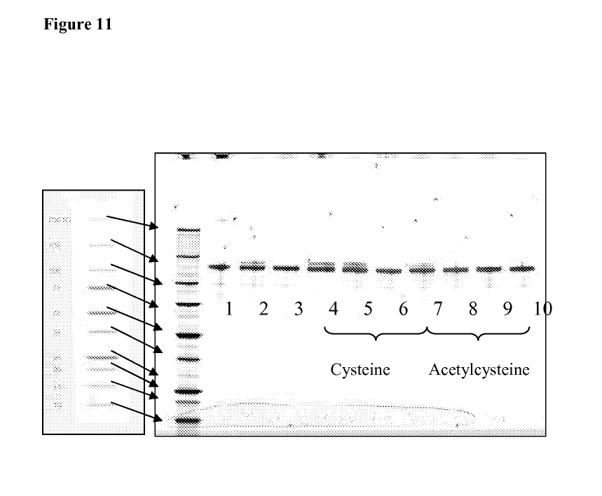

SDS PAGE in non-reducing conditions of the 90 μg dose formulations containing glutathione or monothioglycerol is presented in FIG. 10 and the one with cysteine or N-acetylcysteine is presented in FIG. 11.

FIG. 10 shows SDS-PAGE under non reducing conditions of FINAL BULK stability T15 days 4° C. of the formulations containing glutathione and monothioglycerol. SDS-PAGE legend for FIG. 10:

1 PB

2 CTRL+

3 CTRL−

4 GSH 0.00625%

5 GSH 0.0625%

6 GSH 0.625%

7 MTG 0.00625%

8 MTG 0.0625%

9 MTG 0.625%

10 PB in its Buffer

FIG. 11 shows SDS-PAGE in non reducing conditions of FINAL BULK stability T15 days 4° C. of the formulations containing cysteine and acetylcysteine. SDS-PAGE legend for FIG. 11:

1 PB

2 CTRL+

3 CTRL−

4 Cyst 0.00625%

5 Cyst 0.0625%

6 Cyst 0.625%

7 Acyst 0.00625%

8 Acyst 0.0625%

9 Acyst 0.625%

10 PB in its Buffer

Glutathione, monothioglycerol, cysteine and N-acetylcysteine efficacy during Final Bulk storage 15 days at 4° C. is demonstrated.

Glutathione 0.625%, monothioglycerol 0.625%, cysteine 0.625% and acetyl cysteine 0.625% are at least as efficient as sodium sulfite regarding stability of Final Bulk at 4° C.

In summary F4 formulation comprising cysteine, N-acteyl cysteine or monothioglycerol at a concentration of 0.5% w/v did not show any signs of intermolecular or intramolecular oxidation when stored for 1, 8 or 15 days at 4 degrees C.

F4 formulation comprising glutathione at 0.5% w/v showed no signs of intermolecular or intramolecular oxidation when stored for 1, 8 or 15 days at 4 degrees C.

A corresponding formulation employing sodium sulfite at 0.13% w/v showed some intermolecular oxidation when stored for 1, 8 or 15 days at 4 degrees C.

The formulations of the four chelating agents tested all showed intermolecular and intramolecular oxidation when stored for 24 hours at 4 degrees.

Final Container Stability and Accelerated Stability

Cakes were analyzed after reconstitution in water for injection by SDS PAGE in non-reducing conditions at T0 (time zero) and compared to cakes submitted to accelerated stability (7 day 37° C. and/or AOT [accelerated oxidation testing).

Cakes stored at 37 degrees C. for 7 days showed no signs of intermolecular or intramolecular oxidation when N-acetylcysteine or monothioglycerol were employed at 0.5% w/v. Some intermolecular oxidation was observed when cysteine or glutathione was employed at 0.5% w/v or sodium sulfite was employed at 0.13% w/v.

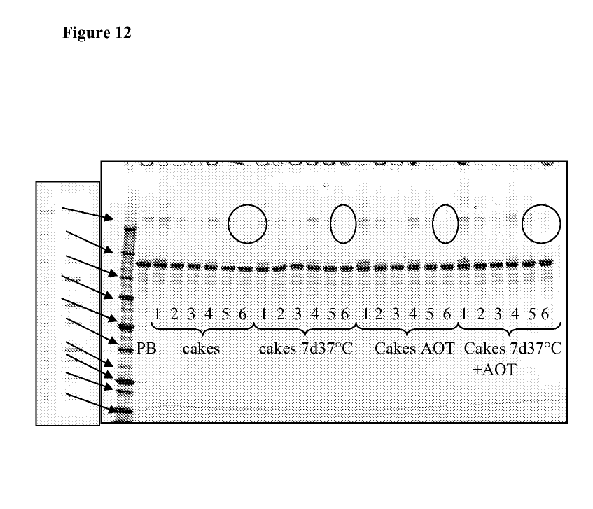

FIG. 12 shows SDS-PAGE in non reducing conditions of reconstituted lyophilized antigen (cakes) containing glutathione and monothioglycerol, where

1 CTRL+

2 CTRL−

3 GSH 0.625%

4 MTG 0.00625%

5 MTG 0.0625%

6 MTG 0.625%

Cakes Subjected to Accelerated Testing

The 4 compounds containing —SH functions are at least as efficient as sodium sulfite even after submission of the cakes to accelerated stability (7 days 37° C., AOT or combination of both). The highest concentration tested (0.5%) of monothioglycerol, cysteine and N-acetylcysteine is more efficient than 10 mM sodium sulfite to avoid the F4co oxidation.

From these data results, conclusion could be drawn that regarding efficacy:
Glutathione 0.5% provided equivalent stabilization to 10 mM Sodium sulfite.
Whereas monothioglycerol 0.5%, cysteine 0.5%, acetylcysteine 0.5% provided superior stabilization 10 mM Sodium sulfite.

F4co Solubility

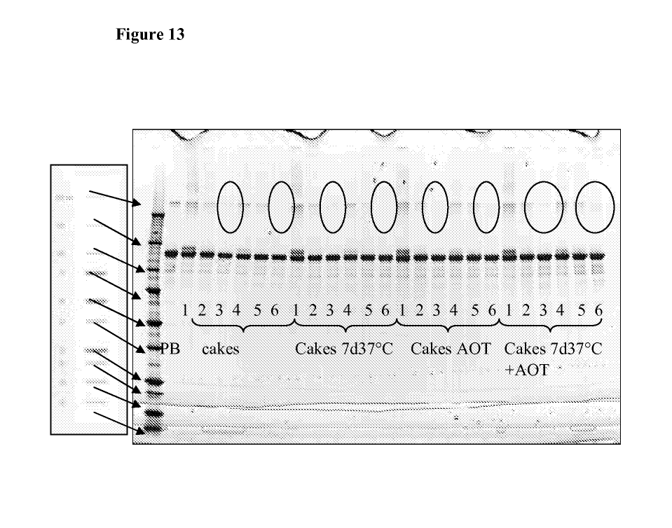

Impact of excipients selected on F4co solubility was investigated 4 hours after reconstitution of cakes in ASO1B. FIG. 13 shows results obtained for cysteine and N-acetylcysteine, where

1 CTRL+

2 CTRL−

3 Cyst 0.625%

4 Acyst 0.00625%

5 Acyst 0.0625%

6 Acyst 0.625%

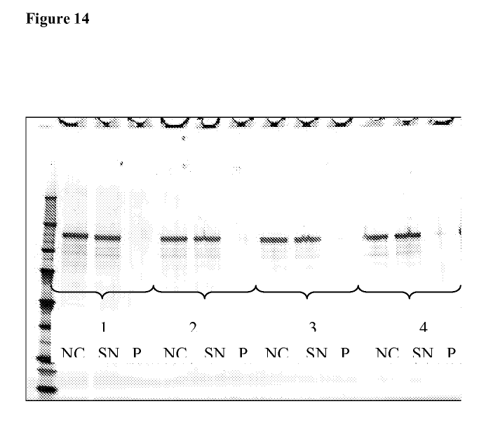

FIG. 14: SDS-PAGE in reducing conditions of reconstituted cakes containing cysteine and acetylcysteine in liposomal adjuvant containing MPL and QS21 after 4 hours at 25° C. (before and after centrifugation), where:

1 CTRL+

2 CTRL−

3 Cyst 0.5%

4 Acyst 0.5%

and where:

NC Non Centrifuged

SN Supernatant

P Pellet

In summary F4 formulation comprising cysteine, N-acteyl cysteine or monothioglycerol at a concentration of 0.5% w/v did not show any signs of intermolecular or intramolecular oxidation when stored with liposomal adjuvant comprising MPL and QS21 for 24 hours at 25 degrees C. F4 formulation comprising glutathione at 0.5% w/v showed some intermolecular oxidation when stored with liposomal adjuvant comprising MPL and QS21 for 24 hours at 25 degrees C. A corresponding formulation employing sodium sulfite at 0.13% w/v showed some intermolecular oxidation when stored at under equivalent conditions.

Formulations with lower amounts of antioxidants showed varying degrees of oxidation.

1. An immunogenic composition comprising:
a) an immunogenic fusion protein comprising Nef or an immunogenic fragment or derivative thereof, and p17 Gag and/or p24 Gag or immunogenic fragments or derivatives thereof, wherein when both p17 and p24 Gag are present there is at least one HIV antigen or immunogenic fragment between them, and
b) an antioxidant containing a thiol functional group.

2. An immunogenic composition of claim 1, wherein the stabilising agent is selected from the group consisting of glutathione, monothioglycerol, cysteine, N-acetyl cysteine or mixtures thereof.

3-5. (canceled)

6. An immunogenic composition of claim 1, wherein the antioxidant is present in a concentration to provide a concentration in the final formulation of about 0.5% w/v.

7. An immunogenic composition of claim 1, which further comprises saccharose, dextrose, mannitol or fructose.

8. An immunogenic composition of claim 7, wherein the saccharose, dextrose, mannitol or fructose is present as 1 to 10% by weight of the final formulation.

9. An immunogenic composition of claim 1, which further comprises arginine.

10. An immunogenic composition of claim 9, wherein the arginine is present in a concentration of 200 to 400 mM.

11. An immunogenic composition of claim 1, which further comprises a chelating agent.

12. An immunogenic composition of claim 11, wherein the chelating agent is selected from citric acid trisodium salt, malic acid sodium salt, dextrose, L-methionine or EDTA disodium.

13-15. (canceled)

16. An immunogenic composition of claim 1, which further comprises a non-ionic surfactant.

17. An immunogenic composition of claim 16, wherein the non-ionic surfactant is Tween 80™.

18. claim 16 wherein the non-ionic surfactant is present at concentration to 0.005 to about 0.05% w/v in a final dose.

19. An immunogenic composition of claim 1, which further comprises a buffer.

20. An immunogenic composition of claim 19 wherein the buffer is a phosphate (PO₄) buffer.

21. (canceled)

22. An immunogenic composition of claim 1 which further comprises a preservative.

23. An immunogenic composition of claim 22, wherein the preservative is thiomersal.

24. An immunogenic composition comprising:
a) an immunogenic fusion protein comprising Nef or an immunogenic fragment or derivative thereof, and p17 Gag and/or p24 Gag or immunogenic fragments or derivatives thereof, wherein when both p17 and p24 Gag are present there is at least one HIV antigen or immunogenic fragment between them,
b) a stabilising agent which is an antioxidant containing a thiol functional group for example selected from the group consisting of glutathione, monothioglycerol, cysteine, N-acetyl cysteine or mixtures thereof,
c) 1% w/v or less of a non-ionic surfactant,
d) 200 to 450 mM of arginine
e) 0.5 to 2.0 mM of a chelating agent,
f) 1 to 50 mM a buffer.

25. (canceled)

26. A pharmaceutical composition comprising the immunogenic composition of claim 1.

27. The pharmaceutical composition of claim 26, which further comprises an adjuvant.

28. The pharmaceutical composition of claim 27, wherein the adjuvant comprises a TLR 4 agonist.

29. The pharmaceutical composition of claim 28, wherein the TRL 4 agonist is MPL.

30. The pharmaceutical composition of claim 27, wherein the adjuvant further comprises a saponin.

31. The pharmaceutical composition of claim 30, wherein the saponin is QS21.

32. The pharmaceutical composition of claim 27, wherein the adjuvant is provided as a liposomal formulation.

33. (canceled)

34. (canceled)

35. A method of treatment comprising administering a therapeutically effective amount of the pharmaceutical composition of claim 26 for the treatment or prophylaxis of HIV or AIDS.

36-38. (canceled)

39. A kit comprising a lyophilized component as defined in claim 25 and a separate container of adjuvant.

40. (canceled)

41. An immunogenic composition of claim 1, wherein the antioxidant is a stabilizing agent.


SEQUENCE LISTING (12 sequences)

SEQ ID NO: 1 - DNA, 3411 bp, Artificial Sequence
  Nucleotide sequence for p24-RT-Nef-p17 fusion protein
atggttatcgtgcagaacatccaggggcaaatggtacatcaggccatatcacctagaactttaaatgcatgggtaaaagtagtagaagagaaggctttcagcccagaagtaatacccatgttttcagcattatcagaaggagccaccccacaagatttaaacaccatgctaaacacagtggggggacatcaagcagccatgcaaatgttaaaagagaccatcaatgaggaagctgcagaatgggatagagtacatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccttttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttgcatatgggccccattagccctattgagactgtgtcagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaaatatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattccttaaaatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtgctagctatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcaggcctatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattactaa

SEQ ID NO: 2 - protein, 1136 aa, Artificial Sequence
  Amino acid sequence for p24-RT-Nef-p17 fusion protein
MVIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLHMGPISPIETVSVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLAMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCRPMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY

SEQ ID NO: 3 - DNA, 3411 bp, Artificial Sequence
  Nucleotide sequence for F4 codon-optimised
atggtcattgttcagaacatacagggccaaatggtccaccaggcaattagtccgcgaactcttaatgcatgggtgaaggtcgtggaggaaaaggcattctccccggaggtcattccgatgttttctgcgctatctgagggcgcaacgccgcaagaccttaataccatgcttaacacggtaggcgggcaccaagccgctatgcaaatgctaaaagagactataaacgaagaggccgccgaatgggatcgagtgcacccggtgcacgccggcccaattgcaccaggccagatgcgcgagccgcgcgggtctgatattgcaggaactacgtctacccttcaggagcagattgggtggatgactaacaatccaccaatcccggtcggagagatctataagaggtggatcatactgggactaaacaagatagtccgcatgtattctccgacttctatactggatatacgccaaggcccaaaggagccgttcagggactatgtcgaccgattctataagacccttcgcgcagagcaggcatcccaggaggtcaaaaattggatgacagaaactcttttggtgcagaatgcgaatccggattgtaaaacaattttaaaggctctaggaccggccgcaacgctagaagagatgatgacggcttgtcagggagtcggtggaccggggcataaagcccgcgtcttacacatgggcccgatatctccgatagaaacagtttcggtcaagcttaaaccagggatggatggtccaaaggtcaagcagtggccgctaacggaagagaagattaaggcgctcgtagagatttgtactgaaatggagaaggaaggcaagataagcaagatcgggccagagaacccgtacaatacaccggtatttgcaataaagaaaaaggattcaacaaaatggcgaaagcttgtagattttagggaactaaacaagcgaacccaagacttttgggaagtccaactagggatcccacatccagccggtctaaagaagaagaaatcggtcacagtcctggatgtaggagacgcatattttagtgtaccgcttgatgaggacttccgaaagtatactgcgtttactataccgagcataaacaatgaaacgccaggcattcgctatcagtacaacgtgctcccgcagggctggaaggggtctccggcgatatttcagagctgtatgacaaaaatacttgaaccattccgaaagcagaatccggatattgtaatttaccaatacatggacgatctctatgtgggctcggatctagaaattgggcagcatcgcactaagattgaggaactgaggcaacatctgcttcgatggggcctcactactcccgacaagaagcaccagaaggagccgccgttcctaaagatgggctacgagcttcatccggacaagtggacagtacagccgatagtgctgcccgaaaaggattcttggaccgtaaatgatattcagaaactagtcggcaagcttaactgggcctctcagatttacccaggcattaaggtccgacagctttgcaagctactgaggggaactaaggctctaacagaggtcatcccattaacggaggaagcagagcttgagctggcagagaatcgcgaaattcttaaggagccggtgcacggggtatactacgacccctccaaggaccttatagccgagatccagaagcaggggcagggccaatggacgtaccagatatatcaagaaccgtttaagaatctgaagactgggaagtacgcgcgcatgcgaggggctcatactaatgatgtaaagcaacttacggaagcagtacaaaagattactactgagtctattgtgatatggggcaagaccccaaagttcaagctgcccatacagaaggaaacatgggaaacatggtggactgaatattggcaagctacctggattccagaatgggaatttgtcaacacgccgccacttgttaagctttggtaccagcttgaaaaggagccgatagtaggggcagagaccttctatgtcgatggcgccgcgaatcgcgaaacgaagctaggcaaggcgggatacgtgactaataggggccgccaaaaggtcgtaacccttacggataccaccaatcagaagactgaactacaagcgatttaccttgcacttcaggatagtggcctagaggtcaacatagtcacggactctcaatatgcgcttggcattattcaagcgcagccagatcaaagcgaaagcgagcttgtaaaccaaataatagaacagcttataaagaaagagaaggtatatctggcctgggtccccgctcacaagggaattggcggcaatgagcaagtggacaagctagtcagcgctgggattcgcaaggttcttgcgatggggggtaagtggtctaagtctagcgtagtcggctggccgacagtccgcgagcgcatgcgacgcgccgaaccagccgcagatggcgtgggggcagcgtctagggatctggagaagcacggggctataacttccagtaacacggcggcgacgaacgccgcatgcgcatggttagaagcccaagaagaggaagaagtagggtttccggtaactccccaggtgccgttaaggccgatgacctataaggcagcggtggatctttctcacttccttaaggagaaaggggggctggagggcttaattcacagccagaggcgacaggatattcttgatctgtggatttaccatacccaggggtactttccggactggcagaattacaccccggggccaggcgtgcgctatcccctgactttcgggtggtgctacaaactagtcccagtggaacccgacaaggtcgaagaggctaataagggcgagaacacttctcttcttcacccggtaagcctgcacgggatggatgacccagaacgagaggttctagaatggaggttcgactctcgacttgcgttccatcacgtagcacgcgagctgcatccagaatatttcaagaactgccgcccaatgggcgccagggccagtgtacttagtggcggagaactagatcgatgggaaaagatacgcctacgcccggggggcaagaagaagtacaagcttaagcacattgtgtgggcctctcgcgaacttgagcgattcgcagtgaatccaggcctgcttgagacgagtgaaggctgtaggcaaattctggggcagctacagccgagcctacagactggcagcgaggagcttcgtagtctttataataccgtcgcgactctctactgcgttcatcaacgaattgaaataaaggatactaaagaggcccttgataaaattgaggaggaacagaataagtcgaaaaagaaggcccagcaggccgccgccgacaccgggcacagcaaccaggtgtcccaaaactactaa

SEQ ID NO: 4 - DNA, 1302 bp, Artificial Sequence
  Nucleotide sequence for P51 RT
atgagtactggtccgatctctccgatagaaacagtttcggtcaagcttaaaccagggatggatggtccaaaggtcaagcagtggccgctaacggaagagaagattaaggcgctcgtagagatttgtactgaaatggagaaggaaggcaagataagcaagatcgggccagagaacccgtacaatacaccggtatttgcaataaagaagaaggattcaacaaaatggcgaaagcttgtagattttagggaactaaacaagcgaacccaagacttttgggaagtccaactaggtatcccacatccagccggtctaaagaagaagaaatcggtcacagtcctggatgtaggagacgcatattttagtgtaccgcttgatgaggacttccgaaagtatactgcgtttactataccgagcataaacaatgaaacgccaggcattcgctatcagtacaacgtgctcccgcagggctggaaggggtctccggcgatatttcagagctctatgacaaaaatacttgaaccattccgaaagcagaatccggatattgtaatttaccaatacatggacgatctctatgtgggctcggatctagaaattgggcagcatcgcactaagattgaggaactgaggcaacatctgcttcgatggggcctcactactcccgacaagaagcaccagaaggagccgccgttcctaaagatgggctacgagcttcatccggacaagtggacagtacagccgatagtgctgcccgaaaaggattcttggaccgtaaatgatattcagaaactagtcggcaagcttaactgggcctctcagatttacccaggcattaaggtccgacagctttgcaagctactgaggggaactaaggctctaacagaggtcatcccattaacggaggaagcagagcttgagctggcagagaatcgcgaaattcttaaggagccggtgcacggggtatactacgacccctccaaggaccttatagccgagatccagaagcaggggcagggccaatggacgtaccagatatatcaagaaccgtttaagaatctgaagactgggaagtacgcgcgcatgcgaggggctcatactaatgatgtaaagcaacttacggaagcagtacaaaagattactactgagtctattgtgatatggggcaagaccccaaagttcaagctgcccatacagaaggaaacatgggaaacatggtggactgaatattggcaagctacctggattccagaatgggaatttgtcaacacgccgccgctggtaaaactgaggcctgctagctaa

SEQ ID NO: 5 - protein, 433 aa, Artificial Sequence
  Amino acid sequence for P51 RT
MSTGPISPIETVSVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLRPAS

SEQ ID NO: 6 - DNA, 1023 bp, Artificial Sequence
  Nef-p17 nucleotide sequence
atgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcaggcctatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattactaa

SEQ ID NO: 7 - protein, 340 aa, Artificial Sequence
  Nef-p17 amino acid sequence
MGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCRPMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY

SEQ ID NO: 8 - DNA, 1029 bp, Artificial Sequence
  P17-Nef nucleotide sequence
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattacctcgacaggcctatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctaa

SEQ ID NO: 9 - DNA, 3411 bp, Artificial Sequence
  Nucleotide sequence for mutated version of F4 where the Methionine at position 592 is replaced by Lysine
atggttatcgtgcagaacatccaggggcaaatggtacatcaggccatatcacctagaactttaaatgcatgggtaaaagtagtagaagagaaggctttcagcccagaagtaatacccatgttttcagcattatcagaaggagccaccccacaagatttaaacaccatgctaaacacagtggggggacatcaagcagccatgcaaatgttaaaagagaccatcaatgaggaagctgcagaatgggatagagtacatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccttttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttgcatatgggccccattagccctattgagactgtgtcagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaaatatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattccttaaaatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcacgtaaacgcggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtgctagctatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcaggcctatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattactaa

SEQ ID NO: 10 - protein, 1136 aa, Artificial Sequence
  Amino acid sequence for mutated version of F4 where the Methionine at position 592 is replaced by Lysine
MVIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLHMGPISPIETVSVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARKRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLAMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCRPMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY

SEQ ID NO: 11 - DNA, 3018 bp, Artificial Sequence
  Nucleotide sequence for mutated F4(p51) where putative internal Methionine initiation site (present in RT portion) replaced by Lysine
atggttatcgtgcagaacatccaggggcaaatggtacatcaggccatatcacctagaactttaaatgcatgggtaaaagtagtagaagagaaggctttcagcccagaagtaatacccatgttttcagcattatcagaaggagccaccccacaagatttaaacaccatgctaaacacagtggggggacatcaagcagccatgcaaatgttaaaagagaccatcaatgaggaagctgcagaatgggatagagtacatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccttttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttgcatatgaggcctggtccgatctctccgatagaaacagtttcggtcaagcttaaaccagggatggatggtccaaaggtcaagcagtggccgctaacggaagagaagattaaggcgctcgtagagatttgtactgaaatggagaaggaaggcaagataagcaagatcgggccagagaacccgtacaatacaccggtatttgcaataaagaagaaggattcaacaaaatggcgaaagcttgtagattttagggaactaaacaagcgaacccaagacttttgggaagtccaactaggtatcccacatccagccggtctaaagaagaagaaatcggtcacagtcctggatgtaggagacgcatattttagtgtaccgcttgatgaggacttccgaaagtatactgcgtttactataccgagcataaacaatgaaacgccaggcattcgctatcagtacaacgtgctcccgcagggctggaaggggtctccggcgatatttcagagctctatgacaaaaatacttgaaccattccgaaagcagaatccggatattgtaatttaccaatacatggacgatctctatgtgggctcggatctagaaattgggcagcatcgcactaagattgaggaactgaggcaacatctgcttcgatggggcctcactactcccgacaagaagcaccagaaggagccgccgttcctaaagatgggctacgagcttcatccggacaagtggacagtacagccgatagtgctgcccgaaaaggattcttggaccgtaaatgatattcagaaactagtcggcaagcttaactgggcctctcagatttacccaggcattaaggtccgacagctttgcaagctactgaggggaactaaggctctaacagaggtcatcccattaacggaggaagcagagcttgagctggcagagaatcgcgaaattcttaaggagccggtgcacagggtatactacgacccctccaaggaccttatagccgagatccagaagcaggggcagggccaatggacgtaccagatatatcaagaaccgtttaagaatctgaagactgggaagtacgcgcgcaaacgaggggctcatactaatgatgtaaagcaacttacggaagcagtacaaaagattactactgagtctattgtgatatggggcaagaccccaaagttcaagctgcccatacagaaggaaacatgggaaacatggtggactgaatattggcaagctacctggattccagaatgggaatttgtcaacacgccgccgctggtaaaactggccctagctatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcaggcctatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattactaa

SEQ ID NO: 12 - protein, 1005 aa, Artificial Sequence
  Amino acid sequence for mutated F4(p51) where putative internal Methionine initiation site (present in RT portion) replaced by Lysine
MVIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLHMRPGPISPIETVSVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARKRGAHTNDVKQLTEAVQKITTESIVIWGKTPKFKLPIQKETWETWWTEYWQATWIPEWEFVNTPPLVKLALAMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCRPMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY